(19) United States
(12) Patent Application Publication
(10) Pub. No.: US 2026/0166177 A1
(43) Pub. Date: Jun. 18, 2026

(54) COMPOSITIONS AND METHODS FOR TREATING AND PREVENTING MACULAR DEGENERATION

(71) Applicant: Genzyme Corporation, Cambridge, MA (US)

(72) Inventor: Abraham SCARIA, Framingham, MA (US)

(73) Assignee: Genzyme Corporation, Cambridge, MA (US)

(21) Appl. No.: 19/372,664

(22) Filed: Oct. 29, 2025

Related U.S. Application Data
(63) Continuation of application No. 17/681,468, filed on Feb. 25, 2022, now Pat. No. 12,483,047.
Application No. 17/681,468 is a continuation of application No. 15/113,720, now abandoned.
(60) Provisional application No. 61/936,797, filed on Feb. 6, 2014.

(51) Int. Cl. A61K 48/00; A61K 9/00; C07K 14/71; H02J 7/02; H02J 7/50; H02J 7/70; H02J 50/10; H02J 50/40

(52) U.S. Cl. CPC A61K 48/005; A61K 9/0048; C07K 14/71; H02J 7/02; H02J 7/50; H02J 7/70; H02J 7/751; H02J 50/10; H02J 50/40; C07K 2319/02; C07K 2319/30; C12N 2750/14143; C12N 2750/14171

(57) ABSTRACT

Compositions and methods for treating macular degeneration are disclosed. The methods utilize gene delivery to human eyes of soluble Flt-1 receptors, as well fusion proteins including a soluble Flt-1 receptor.

### CROSS REFERENCE TO RELATED APPLICATIONS

The present application is a continuation of U.S. patent application Ser. No. 17/681,468, filed Feb. 25, 2022, now U.S. Pat. No. 12,483,047, which is a continuation of U.S. patent application Ser. No. 15/113,720, filed Jul. 22, 2016, which is a National stage application under 35 U.S.C. § 371 of International Application No. PCT/US2015/014872, filed Feb. 6, 2015, which claims priority from U.S. Provisional Application No. 61/936,797, filed Feb. 6, 2014. Each priority application's disclosure is incorporated by reference herein in its entirety.

### SEQUENCE LISTING

The application contains a Sequence Listing which has been submitted electronically in XML format and is hereby incorporated by reference in its entirety. Said .XML copy, created on Apr. 29, 2025, is named “122548.00004.xml” and is 46,049 bytes in size.

### TECHNICAL FIELD

The present invention relates generally to methods for treating and preventing macular degeneration in humans. In particular, the present invention pertains to methods for treating or preventing macular degeneration using the vascular endothelial growth factor (VEGF) receptor, Flt-1.

### SUMMARY OF THE INVENTION

Age-related macular degeneration (AMD) is the primary cause of central irreversible blindness in the elderly. Early clinical presentation of AMD involves subretinal accumulation of debris (drusen). Patients who progress develop either geographic atrophy (GA), with significant degeneration and atrophy of the macular cells, or neovascular AMD (nAMD), with choroidal neovascularization occurring in the end stage of the disease process in an attempt to save the degenerating retina. Blindness results when photoreceptors atrophy following macular retinal pigment epithelial (RPE) degeneration.

Pathogenesis is contingent on aging, environmental and genetic risk factors but the molecular mechanism responsible for disease onset remains largely unknown. The most prominent known genetic factor is a missense mutation residing within the immunoregulatory complement factor H (CFH) gene.

Pathological neovascularization associated with ocular disorders such as nAMD is mediated through the up-regulation of vascular endothelial growth factor (VEGF). Inhibition of VEGF using antibodies, soluble receptors or aptamers has proven to be a promising clinical approach for managing these diseases. While profound improvements in AMD management have been realized, the current anti-VEGF antagonists require repeated intravitreal administrations that can burden both the patient and the treating physician.

Accordingly, there remains a need for developing methods for treating macular degeneration in humans that are less burdensome and commercially viable.

The present invention is based on the discovery that soluble Flt-1 receptors are able to treat macular degeneration in human subjects. Therapeutic results are seen with a wide range of doses when the soluble receptors are delivered using rAAV-mediated gene delivery. High doses were tolerated and yielded therapeutic benefits. In addition, the inventors herein have demonstrated that intravitreal delivery of a single dose as low as 2×10⁸ vector genomes (vg), as well as 2×10¹⁰ vg, resulted in a significant reduction of subretinal and intraretinal fluid two months after injection.

Accordingly, in one embodiment, the invention is directed to a method of treating macular degeneration in a human subject comprising administering to the diseased eye of the subject a composition comprising a recombinant adeno-associated virus (rAAV) virion comprising a polynucleotide encoding a soluble protein comprising at least one domain of vascular endothelial growth factor receptor-1 (VEGFR-1 or Flt-1) capable of modulating VEGF activity, wherein from about 1×10⁷ to about 1×10¹³ rAAV virions are delivered to the eye.

In further embodiments, the invention is directed to a method of treating macular edema in a human subject comprising administering to the diseased eye of the subject a composition comprising a recombinant adeno-associated virus (rAAV) virion comprising a polynucleotide encoding a soluble protein comprising at least one domain of VEGFR-1 (Flt-1) capable of modulating VEGF activity, wherein from about 1×10⁷ to about 1×10¹³ rAAV virions are delivered to the eye.

In embodiments of the above methods, from about 1×10⁷ to about 1×10¹²; 1×10⁸ to about 1×10¹²; about 1×10⁸ to about 1×10¹¹; about 1×10⁸ to about 1×10¹⁰; about 1×10⁸ to about 1×10⁹; about 2×10⁷ to about 2×10¹²; about 2×10⁸ to about 2×10¹²; about 2×10⁸ to about 2×10¹¹; about 2×10⁸ to about 2×10¹⁰; about 2×10⁸ to about 2×10⁹; 2×10⁹ to about 2×10¹⁰; about 1×10¹⁰ to about 1×10¹³; about 1×10¹⁰ to about 1×10¹²; about 1×10¹⁰ to about 1×10¹¹; about 2×10¹⁰ to about 1×10¹³, 2×10¹⁰ to about 1×10¹²; about 2×10¹⁰ to about 2×10¹²; about 2×10¹⁰ to about 1×10¹¹, or about 2×10¹⁰ to about 2×10¹¹ rAAV virions are administered to the eye. In some embodiments, about 1×10⁷, about 2×10⁷, about 6×10⁷, about 1×10⁸, about 2×10⁸, about 6×10⁸, about 1×10⁹, about 2×10⁹, about 6×10⁹, about 1×10¹⁰, about 2×10¹⁰, about 6×10¹⁰, about 1×10¹¹, about 2×10¹¹, about 6×10¹¹, about 1×10¹², about 2×10¹², about 6×10¹², or about 1×10¹³ rAAV virions are administered to the eye.

In additional embodiments, the invention is directed to a method of treating macular degeneration in a human subject comprising administering to the diseased eye of the subject a composition comprising a recombinant adeno-associated virus (rAAV) virion comprising a polynucleotide encoding a soluble protein comprising at least one domain of VEGFR-1 (Flt-1) capable of modulating VEGF activity, wherein less than about 2×10¹⁰ rAAV virions are delivered to the eye.

In further embodiments, the invention is directed to a method of treating macular edema in a human subject comprising administering to the diseased eye of the subject a composition comprising a recombinant adeno-associated virus (rAAV) virion comprising a polynucleotide encoding a soluble protein comprising at least one domain of VEGFR-1 (Flt-1) capable of modulating VEGF activity, wherein less than about 2×10¹⁰ rAAV virions are delivered to the eye.

In any of the methods above, the composition may further comprise an ophthalmologically acceptable vehicle.

In additional embodiments of the above methods, a single intravitreal injection of rAAV virions is administered to the eye.

In further embodiments, the soluble protein comprises:
(a) the at least one domain of Flt-1;
(b) a multimerization domain derived from an immunoglobulin heavy chain; and
(c) a linker 5-25 amino acid residues in length linking (a) to (b),
wherein when the soluble protein is expressed, a multimer of the soluble protein is produced.

In any of the methods above, at least one domain comprises domain 2 of Flt-1.

In further embodiments, the multimer is a homodimer.

In additional embodiments, the multimerization domain comprises the Fc region of an IgG, or an active fragment thereof.

In certain embodiments of the methods above, the multimerization domain comprises the CH3 domain of an IgG, or an active fragment thereof.

In further embodiments, the multimerization domain is from an IgG1, an IgG2, an IgG3 or an IgG4, such as from the constant region of an IgG1 heavy chain.

In additional embodiments, the linker is selected from the group consisting of:

(SEQ ID NO: 1)
gly₉;
(SEQ ID NO: 2)
glu₉;
(SEQ ID NO: 3)
ser₉;
(SEQ ID NO: 4)
gly₅cyspro₂cys;
(SEQ ID NO: 5)
(gly₄ser)₃;
(SEQ ID NO: 6)
SerCysValProLeuMetArgCysGlyGlyCysCysAsn;
(SEQ ID NO: 7)
ProSerCysValProLeuMetArgCysGlyGlyCysCysAsn;
(SEQ ID NO: 8)
GlyAspLeuIleTyrArgAsnGlnLys;
and
(SEQ ID NO: 9)
Gly₉ProSerCysValProLeuMetArgCysGlyGlyCysCysAsn.

In other embodiments, the soluble protein has the formula X-Y-Z, wherein X comprises the IgG-like domain 2 of Flt-1, wherein Y is Gly₉ (SEQ ID NO: 1), and wherein Z is an IgG Fc region or an IgG CH3 region.

In additional embodiments, the multimerization domain is humanized.

In further embodiments, the soluble protein comprises an amino acid sequence selected from the group consisting of (a) the amino acid sequence depicted in FIGS. 2A-2B (SEQ ID NO:11); (b) the amino acid sequence depicted in FIG. 6 (SEQ ID NO:15); (c) the amino acid sequence depicted in FIG. 8 (SEQ ID NO:17); (d) the amino acid sequence depicted in FIG. 12 (SEQ ID NO:21); and (e) an active variant of (a), (b), (c) or (d) having at least 90% sequence identity thereto.

In embodiments of any of the methods above for treating macular degeneration, the macular degeneration is age-related macular degeneration (AMD), such as wet AMD.

In further embodiments of the methods above, the method comprises reducing intraocular pressure, retinal thickness, subretinal fluids, intraretinal fluids, or the like.

In additional embodiments of any of the methods above, the rAAV virion is derived from an AAV serotype selected from AAV1, AAV2, AAV3, AAV4, AAV5, AAV6, AAV7, AAV8, AAV9, AAVAAVrh8, AAVrh8R, AAV10, AAVrh10, AAV11 or AAV12.

In embodiments of any of the methods above, from about 2×10⁸ to less than 2×10¹⁰ rAAV virions are delivered to the eye, such as up to about 2×10⁸ rAAV virions, or up to about 2×10⁹ rAAV virions.

These and other embodiments of the subject invention will readily occur to those of skill in the art in view of the disclosure herein.

### BRIEF DESCRIPTION OF THE FIGURES

FIG. 1 (SEQ ID NO:10) shows the DNA sequence for a fusion protein including Flt-1, termed “sFLT01 protein” herein.

FIGS. 2A-2B (SEQ ID NO:11) show the amino acid sequence for the sFLT01 protein.

FIG. 3 (Genbank accession no. NM003376) (SEQ ID NO:12) shows a DNA sequence encoding VEGF.

FIG. 4 (Genbank accession no. CAC19513) (SEQ ID NO:13) shows an amino acid sequence for VEGF.

FIG. 5 (SEQ ID NO:14) shows the DNA sequence for an additional fusion protein including a soluble Flt-1 linked by a Gly₉ linker to the VEGF multimerization domain, Ex3.

FIG. 6 (SEQ ID NO:15) shows the amino acid sequence encoded by the DNA sequence of FIG. 5 (SEQ ID NO:14).

FIG. 7 (SEQ ID NO:16) shows the DNA sequence for an additional fusion protein including a soluble Flt-1 linked by Gly₉ to the VEGF multimerization domain, Ex3 and a sequence from the IgG1 CH3 region.

FIG. 8 (SEQ ID NO:17) shows the amino acid sequence encoded by the DNA sequence of FIG. 7 (SEQ ID NO:16).

FIGS. 9A-9B (Genbank Accession no. NM_002019) (SEQ ID NO:18) show the DNA sequence encoding for a representative Flt-1 receptor protein.

FIGS. 10A-10E (Genbank accession no. P17948) (SEQ ID NO:19) show the amino acid sequence, of a representative Flt-1 receptor protein.

FIG. 11 (SEQ ID NO:20) shows the DNA sequence for a fusion protein including Flt-1, termed “sFLT02 protein” herein which includes a soluble Flt-1 linked by Gly₉ (SEQ ID NO: 1) to a sequence from the IgG1 CH3 region.

FIG. 12 (SEQ ID NO:21) shows the amino acid sequence for the sFLT02 protein.

FIG. 13 (Genbank accession no Y14737) (SEQ ID NO:22) shows the nucleotide sequence of the IgG1 lambda heavy chain.

FIGS. 14A-14B (SEQ ID NO:23) shows the amino acid sequence of the IgG1 lambda heavy chain.

FIGS. 15A-15B show the changes from baseline (FIG. 15A) (as measured by optical coherence tomography) in subretinal and intraretinal fluid in a human eye treated with a single dose of 2×10⁸ rAAV2-sFLT01 (FIG. 15B).

FIGS. 16A-16B show the changes from baseline (FIG. 16A) (as measured by optical coherence tomography) in subretinal and intraretinal fluid in a human eye treated with a single dose of 2×10¹⁰ rAAV2-sFLT01 (FIG. 16B).

### DETAILED DESCRIPTION OF THE INVENTION

The practice of the present invention will employ, unless otherwise indicated, conventional methods of chemistry, biochemistry, recombinant DNA techniques and immunology, within the skill of the art. Such techniques are explained fully in the literature. See, e.g., Fundamental Virology, 2nd Edition, vol. I & II (B. N. Fields and D. M. Knipe, eds.); Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company, 1993); A. L. Lehninger, Biochemistry (Worth Publishers, Inc., current edition); Methods In Enzymology (S. Colowick and N. Kaplan eds., Academic Press, Inc.); Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011).

All publications, patents and patent applications, and accession numbers cited herein, whether supra or infra, are hereby incorporated by reference in their entirety.

### 1. Definitions

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “an Flt-1 receptor” includes a mixture of two or more such receptors, and the like.

As used herein, “age-related macular degeneration” or “AMD” includes early, intermediate, and advanced AMD and includes both dry AMD such as geographic atrophy and wet AMD, also known as neovascular or exudative AMD. These conditions are described more fully below.

As used herein, “macular edema” refers to the accumulation of fluid within the retina that can cause swelling or thickening of the macular area of the eye. Macular edema develops when blood vessels in the retina leak fluids. Pathophysiology typically involves vascular instability and a breakdown of the blood-retinal barrier. Cystoid macular edema (CME), the most common type observed, involves fluid accumulation in the outer plexiform layer secondary to abnormal perifoveal retinal capillary permeability. The macula does not function properly when it is swollen. Vision loss may be mild to severe, but in some cases, peripheral vision remains.

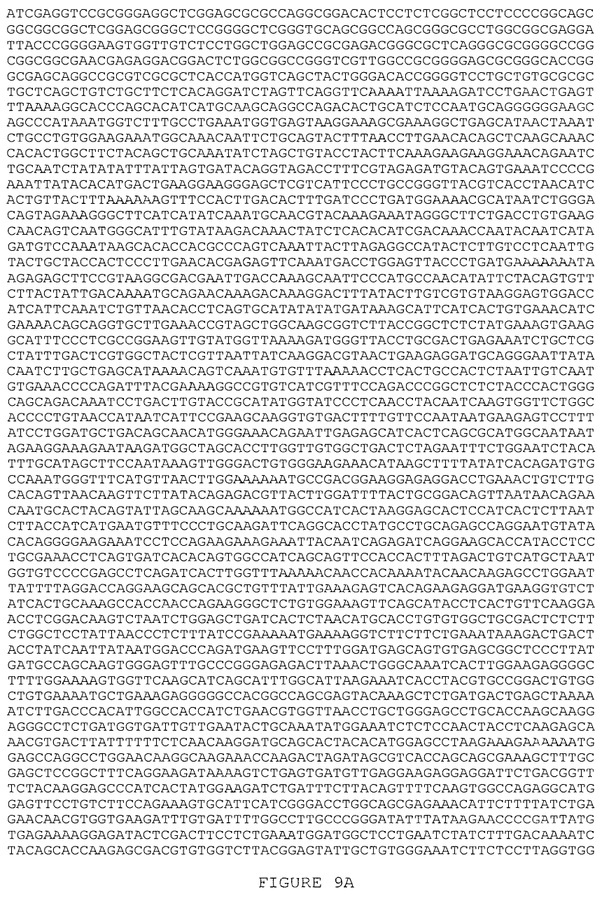

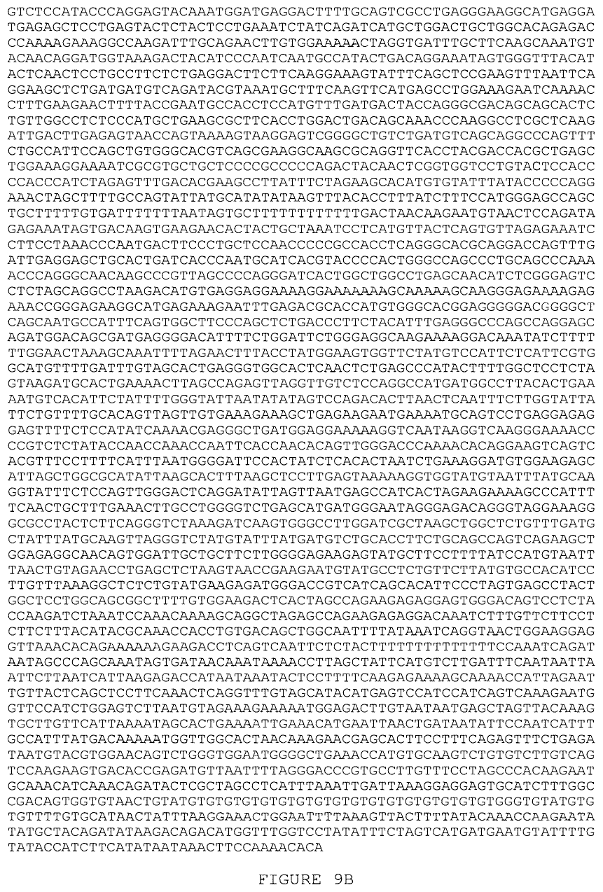

The terms “Flt-1 protein” and “VEGF-R1 protein” are used interchangeably herein and denote a receptor protein known to bind VEGF. The terms “Flt-1 protein” and “VEGF-R1 protein” or a nucleotide sequence encoding the same, refer to a protein or nucleotide sequence, respectively, that is derived from any Flt-1 protein, regardless of source. The terms, as used herein, refer to molecules capable of binding to and modulating activity of VEGF, as measured in any of the known VEGF activity tests, including those described further herein. The full-length nucleotide sequence and corresponding amino acid sequence of a representative Flt-1 protein are shown in FIGS. 9A-9B (SEQ ID NO:18) and 10A-10E (SEQ ID NO:19), respectively. However, an Flt-1 protein as defined herein is not limited to the depicted sequences as several such receptors are known and variations in these receptors will occur between species. Non-limiting examples of additional Flt-1 protein sequences can be found in GenBank Accession Nos. AF063657.2; BC039007.1; U01134.1; HD077716.1; X51602.1; EU360600.1; AK300392.1; EU826561.1; EU368830.1; AB385191.1; AK292936.1; AK309901.1; AB209050.1; BC029849.1; BC039007.1; NM_001160031.1; NM_001160030.1; NM_002019.4; NM_001159920.1.

The full-length proteins, with or without the signal sequence, and fragments thereof, as well as proteins with modifications, such as deletions, additions and substitutions (either conservative or non-conservative in nature), to the native sequence, are intended for use herein, so long as the protein maintains the desired activity. Such active variants and fragments are considered VEGF1 receptors in the context of the present invention. Modifications may be deliberate, as through site-directed mutagenesis, or may be accidental, such as through mutations of hosts which produce the proteins or errors due to PCR amplification. Accordingly, active proteins substantially homologous to the parent sequence, e.g., proteins with 70 . . . 80 . . . 85 . . . 90 . . . 95 . . . 98 . . . 99% etc. identity that retain the ability to modulate activity of the corresponding ligand, are contemplated for use herein.

A “native” polypeptide, such as an Flt-1 receptor, refers to a polypeptide having the same amino acid sequence as the corresponding molecule derived from nature. Such native sequences can be isolated from nature or can be produced by recombinant or synthetic means. The term “native” sequence specifically encompasses naturally-occurring truncated or secreted forms of the specific molecule (e.g., an extracellular domain sequence), naturally-occurring variant forms (e.g., alternatively spliced forms) and naturally-occurring allelic variants of the polypeptide. In various embodiments of the invention, the native molecules disclosed herein are mature or full-length native sequences comprising the full-length amino acids sequences shown in the accompanying figures. However, while some of the molecules disclosed in the accompanying figures begin with methionine residues designated as amino acid position 1 in the figures, other methionine residues located either upstream or downstream from amino acid position 1 in the figures may be employed as the starting amino acid residue for the particular molecule. Alternatively, depending on the expression system used, the molecules described herein may lack an N-terminal methionine.

By “extracellular domain” is meant a form of the receptor polypeptide which includes all or a fragment of the extracellular domain and lacks all or a portion of the transmembrane domain and may also be devoid of the cytoplasmic domain. Typically, when used in the present invention, the extracellular domain is essentially free of both the transmembrane and cytoplasmic domains. Ordinarily, an extracellular domain includes less than 10% of such transmembrane and/or cytoplasmic domains, less than 5% of these domains, less than 1%, or less than 0.5% of such domains. Transmembrane domains for the receptors described herein can be identified pursuant to criteria routinely employed in the art for identifying hydrophobic domains, for example, using standard hydropathy plots, such as those calculated using the Kyte-Doolittle technique, Kyte et al., J. Mol. Biol. (1982) 157:105-132.

As explained above, the receptors for use with the present invention may or may not include the native signal sequence. The approximate location of the signal peptides of the receptors described herein are described in the specification and in the accompanying figures. It is noted, however, that the C-terminal boundary of a signal peptide may vary, typically by no more than about 5 amino acids on either side of the signal peptide C-terminal boundary as described herein. The C-terminal boundary of the signal peptide may be identified pursuant to criteria routinely employed in the art, such as described in Nielsen et al., Prot. Eng. (1997) 10:1-6 and von Heinje et al., Nucl Acids. Res. (1986) 14:4683-4690. Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

By “variant” is meant an active polypeptide as defined herein having at least about 80% amino acid sequence identity with the corresponding full-length native sequence, a polypeptide lacking the signal peptide, an extracellular domain of a polypeptide, with or without a signal peptide, or any other fragment of a full-length polypeptide sequence as disclosed herein. Such polypeptide variants include, for instance, polypeptides wherein one or more amino acid residues are added, or deleted, at the N- and/or C-terminus of the full-length native amino acid sequence. In embodiments, a variant will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to the corresponding full-length native sequence. In embodiments, variant polypeptides are at least about 10 amino acids in length, such as at least about 20 amino acids in length, e.g., at least about 30 amino acids in length, alternatively at least about 40 amino acids in length, alternatively at least about 50 amino acids in length, alternatively at least about 60 amino acids in length, alternatively at least about 70 amino acids in length, alternatively at least about 80 amino acids in length, alternatively at least about 90 amino acids in length, alternatively at least about 100 amino acids in length, alternatively at least about 150 amino acids in length, alternatively at least about 200 amino acids in length, alternatively at least about 300 amino acids in length, or more. Variants include substitutions that are conservative or non-conservative in nature. For example, the polypeptide of interest may include up to about 5-10 conservative or non-conservative amino acid substitutions, or even up to about 15-25 or 50 conservative or non-conservative amino acid substitutions, or any number between 5-50, so long as the desired function of the molecule remains intact.

“Homology” refers to the percent identity between two polynucleotide or two polypeptide moieties. Two DNA, or two polypeptide sequences are “substantially homologous” to each other when the sequences exhibit at least about 50%, at least about 75%, at least about 80%-85%, at least about 90%, at least about 95%-98% sequence identity, at least about 99%, or any percent therebetween over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Methods for determining percent identity are well known in the art. For example, percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, DC, which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, WI) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, CA). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are well known in the art.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

By the term “degenerate variant” is intended a polynucleotide containing changes in the nucleic acid sequence thereof, that encodes a polypeptide having the same amino acid sequence as the polypeptide encoded by the polynucleotide from which the degenerate variant is derived.

A “coding sequence” or a sequence which “encodes” a selected polypeptide, is a nucleic acid molecule which is transcribed (in the case of DNA) and translated (in the case of mRNA) into a polypeptide when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

By “vector” is meant any genetic element, such as a plasmid, phage, transposon, cosmid, chromosome, virus, virion, etc., which is capable of replication when associated with the proper control elements and which can transfer gene sequences to cells. Thus, the term includes cloning and expression vehicles, as well as viral vectors.

By “recombinant vector” is meant a vector that includes a heterologous nucleic acid sequence which is capable of expression in a cell.

A “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one, in embodiments two, inverted terminal repeat sequences (ITRs).

A “recombinant AAV vector (rAAV vector)” refers to a polynucleotide vector comprising one or more heterologous sequences (i.e., nucleic acid sequence not of AAV origin) that are flanked by at least one, in embodiments two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, particularly an AAV particle. A rAAV vector can be packaged into an AAV virus capsid to generate a “recombinant adeno-associated viral particle (rAAV particle)”.

By “recombinant virus” is meant a virus that has been genetically altered, e.g., by the addition or insertion of a heterologous nucleic acid construct into the particle.

The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous molecules into suitable host cells.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A “nucleic acid” sequence refers to a DNA or RNA sequence. The term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil,-uracil-5-oxyacetic acid methylester, uracil-5-oxyacetic acid, pseudouracil, queosine, 2-thiocytosine, and 2,6-diaminopurine.

The term DNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

“Operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The term “multimerization domain” as used in the context of the present invention, is meant to refer to the portion of the molecule to which the particular Flt-1 receptor is joined, either directly or through a “linker domain.” The multimerization domain can be a polypeptide domain which facilitates the interaction of two or more multimerization domains and/or sFlt-1 receptor domains.

For example, a multimerization domain may be an immunoglobulin sequence, such as an immunoglobulin constant region, a leucine zipper, a hydrophobic region, a hydrophilic region, a polypeptide comprising a free thiol which forms an intermolecular disulfide bond between two or more multimerization domains or, for example a “protuberance-into-cavity” domain described in, for example, U.S. Pat. No. 5,731,168, incorporated herein by reference in its entirety. Protuberances are constructed by, e.g., replacing small amino acid side chains from the interface of a first polypeptide with a larger side chain (for example a tyrosine or tryptophan). Compensatory cavities of identical or similar size to the protuberances are optionally created on the interface of a second polypeptide by replacing large amino acid side chains with smaller ones (for example alanine or threonine).

Therefore, in aspects, the multimerization domain provides that portion of the molecule which promotes or allows the formation of dimers, trimers, and the like from monomeric domains. In aspects, multimerization domains are immunoglobulin constant region domains.

“Immunoglobulins” (Igs) are proteins, generally glycoproteins, that are antibodies or antibody-like molecules which lack antigen specificity. Immunoglobulins are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has an amino (N) terminal variable domain (VH) followed by carboxy (C) terminal constant domains. Each light chain has a variable N-terminal domain (VL) and a C-terminal constant domain; the constant domain of the light chain (CL) is aligned with the first constant domain (CH1) of the heavy chain, and the light chain variable domain is aligned with the variable domain of the heavy chain. According to the domain definition of immunoglobulin polypeptide chains, light (L) chains have two conformationally similar domains VL and CL; and heavy chains have four domains (VH, CH1, CH2, and CH3) each of which has one intrachain disulfide bridge.

Depending on the amino acid sequence of the constant (C) domain of the heavy chains, immunoglobulins can be assigned to different classes. There are five major classes of immunoglobulins: IgA, IgD, IgE, IgG, and IgM. The immunoglobulin class can be further divided into subclasses (isotypes), e.g., IgG1, IgG2, IgG3, IgG4, IgG5, IgA1, and IgA2. Each heavy chain has at one end a variable domain (VH) followed by a number of constant domains. The light chains of antibodies from any vertebrate species can be assigned to one of two distinct types called kappa (κ) or lambda (λ), based upon the amino acid sequence of their constant domains.

The term “Fc region” refers to the C-terminal (constant) region of an immunoglobulin heavy chain. The Fc region may be a native sequence Fc region or a variant Fc region. Although the boundaries of the Fc region of an immunoglobulin heavy chain may vary, the human IgG heavy chain Fc region may stretch from an amino acid residue at position Cys226, or from Pro230, to the carboxyl-terminus of a full-length human IgG1. The Fc region of an immunoglobulin generally comprises two constant domains, CH2 and CH3. The last residue, lysine, in the heavy chain of IgG1 can but need not be present as the terminal residue in the Fc in the mature protein. One human IgG1 heavy chain Fc region is defined in NCBI accession number P01857.

The “CH2 domain” of a human IgG1 Fc region (also referred to as “Cy2” domain) usually extends from about amino acid 231 to about amino acid 340 of a full-length IgG, but from Pro111 to Lys223 of the human IgG heavy chain Fc region.

The “CH3 domain” comprises the residues C-terminal to a CH2 domain in a human IgG1 Fc region (i.e. from about amino acid residue 341 to about amino acid residue 447 of a full-length IgG, but from Gly224 to Lys330 of a human IgG heavy chain Fc region).

The “hinge region” is generally defined as stretching from Glu216 to Pro230 of a full-length human IgG1 (Burton, Molec. immunol. (1985) 22:161-206), but from Glu99 to Pro110 of a human IgG heavy chain Fc region. Hinge regions of other IgG isotypes may be aligned with the IgG1 sequence by placing the first and last cysteine residues forming inter-heavy chain S—S bonds in the same positions.

The “lower hinge region” of an Fc region is normally defined as the stretch of residues immediately C-terminal to the hinge region, i.e. residues 233 to 239 of a full-length human IgG1.

A “native Fc region sequence” comprises an amino acid sequence identical to the amino acid sequence of an Fc region found in nature. Native human Fc region sequences include but are not limited to the human IgG1 Fc region (non-A and A allotypes); the human IgG2 Fc region; the human IgG3 Fc region; and the human IgG4 Fc region as well as naturally occurring variants thereof. Native Fc regions from other species, such as murine Fc regions, are also well known.

A “functional Fc region” possesses an “effector function” of a native Fc region. Exemplary “effector functions” include C1q binding; complement-dependent cytotoxicity; Fc receptor binding; antibody-dependent cell-mediated cytotoxicity (ADCC); phagocytosis; down regulation of cell surface receptors (e.g., B cell receptor; BCR), etc. Such effector functions typically require the Fc region to be combined with a binding domain (i.e., a VEGF ligand herein) and can be assessed using various assays known in the art. The Fc region can be a human Fc region, e.g. a native sequence human Fc region such as a human IgG1 (A and non-A allotypes), IgG2, IgG3 or IgG4 Fc region. Such sequences are known. See, e.g., PCT Publication NO. WO01/02440, incorporated herein by reference in its entirety.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome (e.g., transcribed into a molecule that confers a desired therapeutic or diagnostic outcome).

The terms “genome particles (gp),” “genome equivalents,” or “genome copies” as used in reference to a viral titer, refer to the number of virions containing the recombinant AAV DNA genome, regardless of infectivity or functionality. The number of genome particles in a particular vector preparation can be measured by procedures such as described in the Examples herein, or for example, in Clark et al. (1999) Hum. Gene Ther., 10:1031-1039; Veldwijk et al. (2002) Mol. Ther., 6:272-278.

The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in Mclaughlin et al. (1988) J. Virol., 62:1963-1973.

The term “transducing unit (tu)” as used in reference to a viral titer, refers to the number of infectious recombinant AAV vector particles that result in the production of a functional transgene product as measured in functional assays such as described in Examples herein, or for example, in Xiao et al. (1997) Exp. Neurobiol., 144:113-124; or in Fisher et al. (1996) J. Virol., 70:520-532 (LFU assay).

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contain several shorter regions of self-complementarity (designated A, A′, B, B′, C, C′ and D regions), allowing intrastrand base-pairing to occur within this portion of the ITR.

A “terminal resolution sequence” or “trs” is a sequence in the D region of the AAV ITR that is cleaved by AAV rep proteins during viral DNA replication. A mutant terminal resolution sequence is refractory to cleavage by AAV rep proteins.

A “helper virus” for AAV refers to a virus that allows AAV (which is a defective parvovirus) to be replicated and packaged by a host cell. A helper virus provides “helper functions” which allow for the replication of AAV. A number of such helper viruses have been identified, including adenoviruses, herpesviruses and poxviruses such as vaccinia. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include E1A functions, E1B functions, E2A functions, VA functions and E4 or f6 functions.

A preparation of rAAV is said to be “substantially free” of helper virus if the ratio of infectious AAV particles to infectious helper virus particles is at least about 10²:1; at least about 10⁴:1, at least about 10⁶:1; or at least about 10⁸:1. Preparations can also be free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

The term “modulate” means to affect (e.g., either upregulate, downregulate or otherwise control) the level of a signaling pathway. Cellular processes under the control of signal transduction include, but are not limited to, transcription of specific genes, normal cellular functions, such as metabolism, proliferation, differentiation, adhesion, apoptosis and survival, as well as abnormal processes, such as transformation, blocking of differentiation and metastasis.

“Active” or “activity” for purposes of the present invention refers to forms of an Flt-1 receptor polypeptide which retain a biological activity (either inhibitory or stimulatory) of the corresponding native or naturally occurring polypeptide. The activity may be greater than, equal to, or less than that observed with the corresponding native or naturally occurring polypeptide. As explained above, an activity includes modulating the level of the VEGF signaling pathways in a subject suffering from macular degeneration.

By “isolated” when referring to a nucleotide sequence, is meant that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

For the purpose of describing the relative position of nucleotide sequences in a particular nucleic acid molecule throughout the instant application, such as when a particular nucleotide sequence is described as being situated “upstream,” “downstream,” “3-prime (3′)” or “5-prime (5′)” relative to another sequence, it is to be understood that it is the position of the sequences in the “sense” or “coding” strand of a DNA molecule that is being referred to as is conventional in the art.

The term “purified” refers to isolation of a substance (compound, polynucleotide, protein, polypeptide, polypeptide composition) such that the substance of interest comprises the majority percent of the sample in which it resides. Typically in a sample a substantially purified component comprises 50%, 80%-85%, 90-99%, such as at least 90%, 91%, 92%, 93%, 94%, 95%, 96%, 97%, 98%, 99% of the sample. Techniques for purifying polynucleotides and polypeptides of interest are well-known in the art and include, for example, ion-exchange chromatography, affinity chromatography and sedimentation according to density.

The terms “subject”, “individual” or “patient” are used interchangeably herein and refer to a vertebrate, e.g., a mammal. Mammals include, but are not limited to, murines, rodents, simians, humans, farm animals, sport animals and pets.

The terms “effective amount” or “therapeutically effective amount” of a composition or agent, as provided herein, refer to a sufficient amount of the composition or agent to provide the desired response, such as modulating VEGF in the eye, or reducing, preventing or retarding progression of the physical changes in the eye related to macular degeneration, or reducing, preventing or retarding progression of the symptoms manifested therefrom (e.g., accumulation of drusen, abnormal blood vessel growth in the eye, abnormal fluid, blood and protein leakage in the eye, and the like). The exact amount required will vary from subject to subject, depending on the species, age, and general condition of the subject, the severity of the condition being treated, and the particular macromolecule of interest, mode of administration, and the like. An appropriate “effective” amount in any individual case may be determined by one of ordinary skill in the art using routine experimentation. See, e.g., Lim, J. (2012) Age-Related Macular Degeneration, CRC Press, Boca Raton; Kanski et al. (2011) Clinical Ophthalmology: A Systematic Approach, Elsevier Saunders.

“Treatment” or “treating” macular degeneration includes: (1) preventing the disease, i.e., preventing the development of the disease or causing the disease to occur with less intensity in a subject that may be exposed to or predisposed to the disease but does not yet experience or display symptoms of the disease, (2) inhibiting the disease, i.e., arresting the development, preventing or retarding progression, or reversing the disease state (3) relieving symptoms of the disease i.e., decreasing the number of symptoms experienced by the subject, or (4) reducing, preventing or retarding progression of the physical changes in the eye related to macular degeneration. Treatment includes, but is not limited to, reduction in accumulation of drusen, abnormal blood vessel growth in the eye, abnormal fluid, blood and protein leakage in the eye, and the like. Treatment can be detected, for example, by monitoring the rate and amount of loss of photoreceptors (rods and cones) in the central part of the eye, by monitoring the rate of vision loss and the best corrected visual acuity (BCVA), by monitoring the rate and amount of atrophy of the retinal pigment epithelial layer (and the choriocapillaris) below the retina, by monitoring the amount of drusen (cellular debris) that accumulates between the retina and the choroid, by monitoring abnormal blood vessel growth in the eye, and monitoring the amount of abnormal fluid, blood and protein leakage in the eye.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present invention, exemplary methods, devices, and materials are now described. All technical and patent publications cited herein are incorporated herein by reference in their entirety. Nothing herein is to be construed as an admission that the invention is not entitled to antedate such disclosure by virtue of prior invention.

It is to be understood, although not always explicitly stated that all numerical designations are preceded by the term “about.” It also is to be understood, although not always explicitly stated, that the reagents described herein are merely exemplary and that equivalents of such are known in the art.

### 2. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

It should be appreciated that the invention should not be construed to be limited to the examples described herein. Methods and materials similar or equivalent to those described herein can be used in the practice of the present invention, and the invention should be construed to include any and all applications provided herein and all equivalent variations within the skill of the ordinary artisan.

Central to the present invention is the discovery that gene delivery to the human eye, using constructs encoding a soluble protein comprising at least one domain of VEGFR-1 (Flt-1) capable of modulating VEGF activity (also termed “a soluble Flt-1 protein” or “soluble Flt-1 receptor” herein), serves to modulate the corresponding signaling pathways, and significantly reduces symptoms of macular degeneration. In aspects, the invention involves administering doses lower than that previously reported as efficacious in non-human primates. See, e.g., Lukason et al., Molecular Ther. (2011) 19:260-265. Thus, administration of soluble Flt-1 proteins provides a useful technique for treating and preventing macular degeneration in humans. The methods described herein can be used alone or in combination with traditional therapies (e.g., PDGF antagonists, PDGF-R antagonists, complement pathway inhibitors).

In embodiments, the soluble protein used in the present methods is a fusion protein that includes at least one Flt-1 domain, or an active portion thereof, linked to a multimerization domain, either directly or via a linker, such as linked to an immunoglobulin constant region. In some embodiments, the soluble protein includes domain 2 or portions and/or extensions thereof, linked to a multimerization domain, either directly or via a linker. Linkers can include sequences of amino acids 5-25 residues in length. Representative multimerization domains include, but are not limited to, an IgG Fc region, or portions thereof, and an IgG CH3 region, or portions thereof.

The receptor can be present either upstream or downstream from the immunoglobulin region. Typically, the fusion protein is produced in multimeric form when expressed in vivo. The multimer can be a dimer, trimer, etc.

In order to further an understanding of the invention, a more detailed discussion is provided below regarding macular degeneration, Flt-1 receptors, receptor-immunoglobulin fusions, as well as various gene delivery methods for use with the present invention.

### Macular Degeneration

As explained above, the present invention makes use of Flt-1 receptors in order to inhibit VEGF activity and thereby treat, prevent, alleviate, and/or prevent or retard progression of macular degeneration. In certain embodiments, an individual at risk of developing macular degeneration is administered an amount effective to delay or prevent the disease.

At least three forms of macular degeneration have been identified. (1) Atrophic, non-exudative-dry form of AMD, also known as central geographic atrophy, occurs in approximately 85 to 90% of patients with macular degeneration. The dry form of AMD typically results from atrophy of the retinal pigment epithelial layer (and presumably the choriocapillaris) below the retina and causes vision loss through loss of photoreceptors (rods and cones) in the central part of the eye. There can additionally be cellular debris (called drusen) accumulating between the retina and the choroid. (2) The wet form of AMD, also known as neovascular or exudative AMD, represents the more severe form of AMD. The wet form of AMD is typically characterized by abnormal blood vessel growth in the eye, wherein the faulty blood vessels leak fluids and blood. It may cause vision loss due to abnormal blood vessel growth from the choriocapillaries through Bruch's membrane into the subretinal space, ultimately leading to blood and protein leakage below the macula. Bleeding, leaking, and scarring from these blood vessels eventually causes irreversible damage to the photoreceptors, scar formation in the macula and relatively rapid vision loss if left untreated. (3) Pigment epithelial detachment associated (PED) ARMD occurs in less than 5% of patients and results in retinal detachment.

### Flt-1 Molecules and Fusions

The present invention makes use of soluble forms of Flt-1 receptors to modulate VEGF activity and thereby treat, prevent, alleviate, and/or prevent or retard progression of macular degeneration. In aspects, Flt-1 receptor-immunoglobulin fusions are used in the present invention. The native molecule, as well as active fragments and analogs thereof that retain the ability to bind VEGF and modulate ligand activity, as measured in any of the known various assays and animal models including those described further herein, are suitable for use with the present invention. For example, VEGF binding assays are known and described in Pechan et al., Gene Ther (2009) 16:10-16 and U.S. Pat. No. 7,928,072, incorporated herein by reference in its entirety.

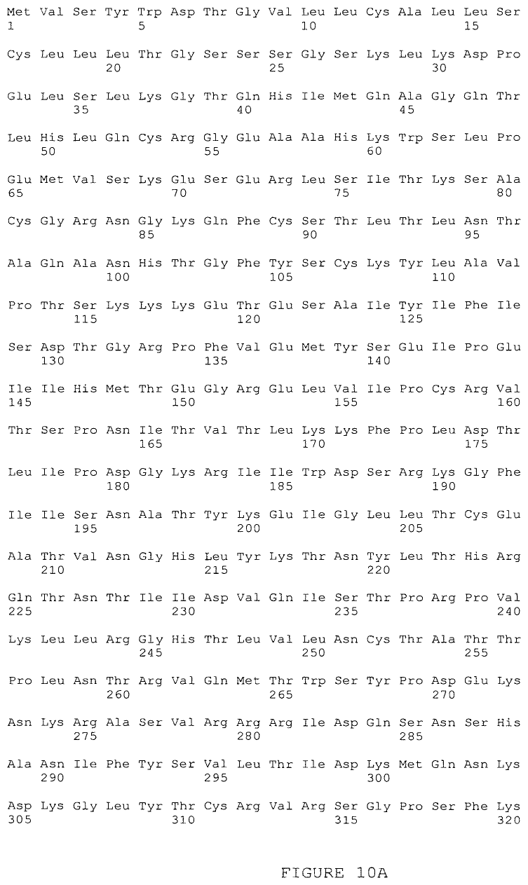

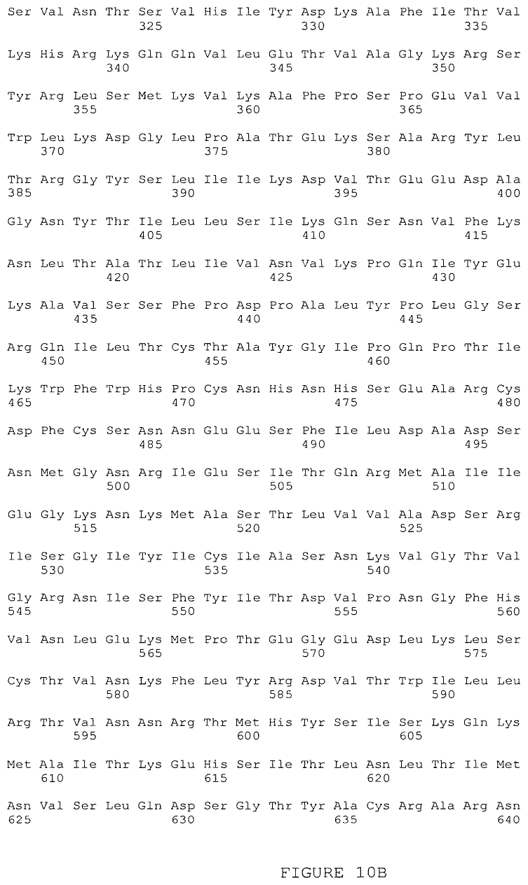

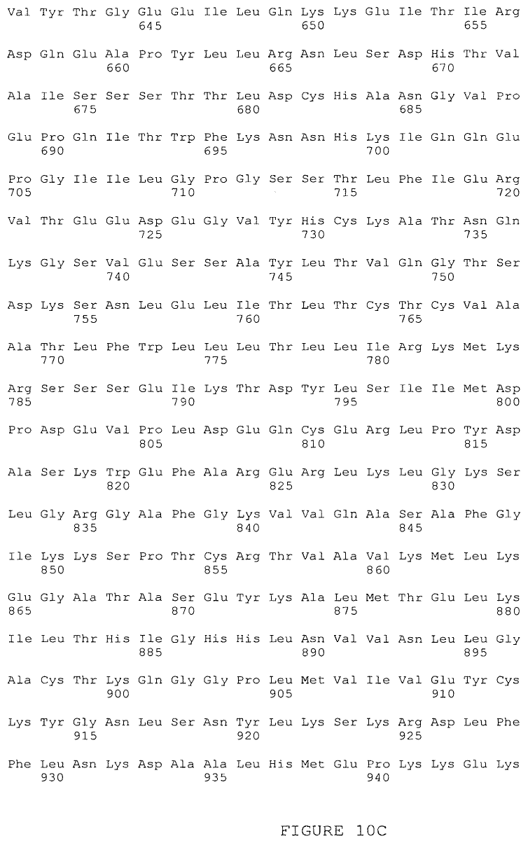

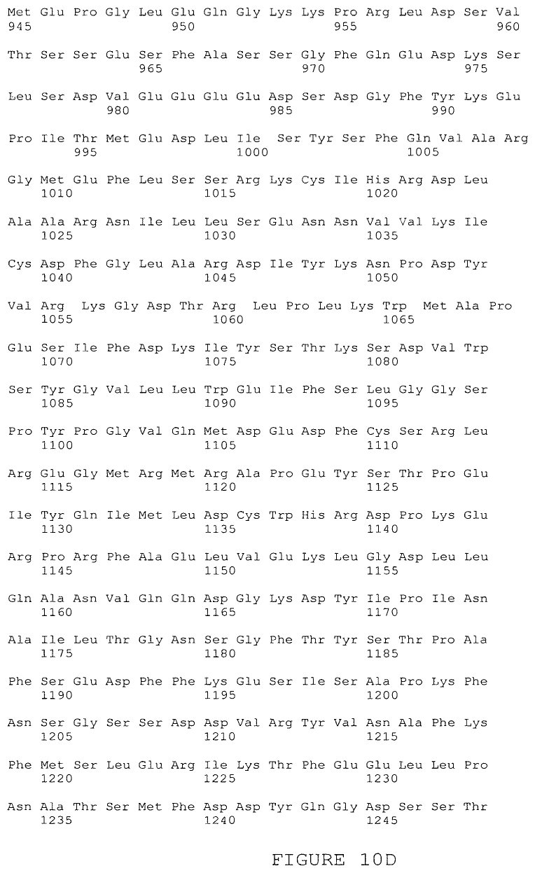

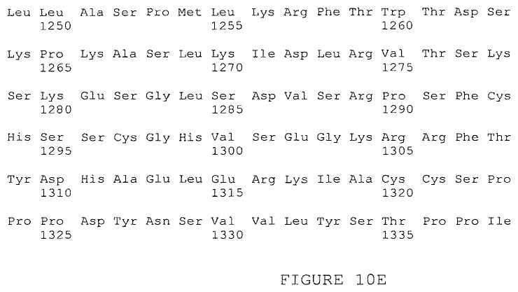

The amino acid sequence and nucleotide sequence encoding for a representative full-length human Flt-1 receptor is shown in FIGS. 9A-9B (SEQ ID NO:18) and 10A-10E (SEQ ID NO: 19), respectively. The Flt-1 receptor protein has an extracellular portion found at positions 27-758 of FIGS. 10A-10E which comprises seven Ig-like domains. Amino acids 1-26 of FIGS. 10A-10E represent a signal sequence. The seven Ig-like domains are located at residue numbers 32-123, 151-214, 230-327, 335-421, 428-553, 556-654, and 661-747, respectively, of FIGS. 10A-10E. This Flt-1 protein is encoded by the DNA sequence shown at Genbank accession no. NM_002019 (FIGS. 9A-9B, SEQ ID NO:18).

In embodiments, the Flt-1 molecules used in the present invention include an Flt-1 Ig-like domain 2. Any portion of the Flt-1 molecule can be used, so long as the molecule retains the ability to modulate VEGF activity; however, in some embodiments, the Flt-1 molecule can lack all or a portion of domains 1 and 3. Flt-1 domain 2 is found at positions 151-214 of FIGS. 10A-10E. However, the Flt-1 component of the present fusions can include, for example, any sequence of amino acids found between domains 1 and 2, domains 2 and 3, etc. of Flt-1, e.g., any sequence of amino acids corresponding to an amino acid sequence found between positions 124-229 of FIGS. 10A-10E, such as an amino acid sequence beginning at any one of positions 124, 125, 126, 127, 128, 129, 130, 131, 132, 133, 134, 135, 136, 136 . . . 140 . . . 145 . . . 150, 151, 152, 153, 154, 155 . . . 160 . . . 165 . . . 170, up to amino acid 210, 211, 212, 213, 214, 215, 216, 217, 218, 219, 220, 221, 222, 223, 224, 225, 226, 227, 228, 229, etc. of FIGS. 10A-10E. In embodiments, the Flt-1 component of the fusions described herein includes amino acids 132-226 of FIGS. 10A-10E. The Flt-1 component can also include portions of any of the other domains present in the extracellular region of the Flt-1 protein, including portions of domains 1 and 3, or even deletions of domain 2, so long as the desired activity is maintained. In certain embodiments, domains 1 and 3 are not present in their entireties.

Moreover, the soluble proteins of the invention can include additional polypeptide/moieties. For example, the soluble proteins of the invention can include all or portions of VEGFR2, such as any of the various domains of VEGFR2, including without limitation domains 1, 2 and/or 3 of VEGFR2, as well as constructs with one or more, or portions of these domains deleted. See, e.g. Holash et al., Proc. Natl. Acad. Sci. USA (2002) 99:11393-11398 and U.S. Pat. No. 7,378,095, incorporated herein by reference in its entirety, for descriptions of VEGFR2 fusions and hybrid fusions of domains from VEGFR2 with Flt-1 domains.

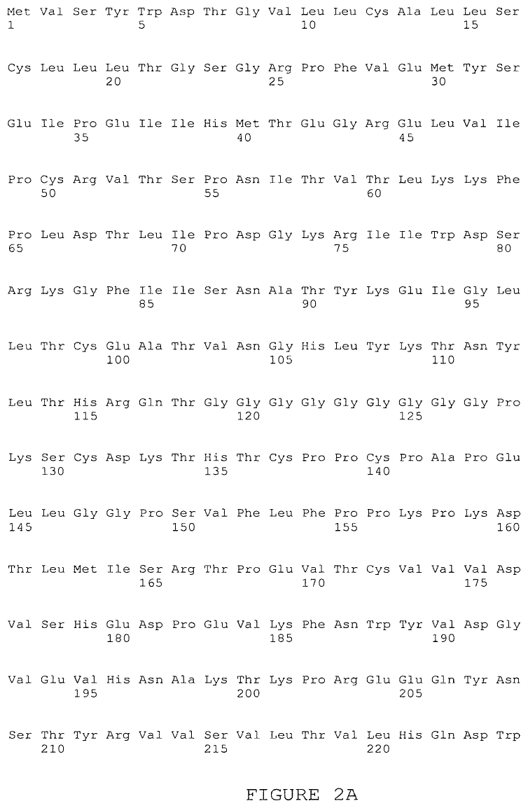

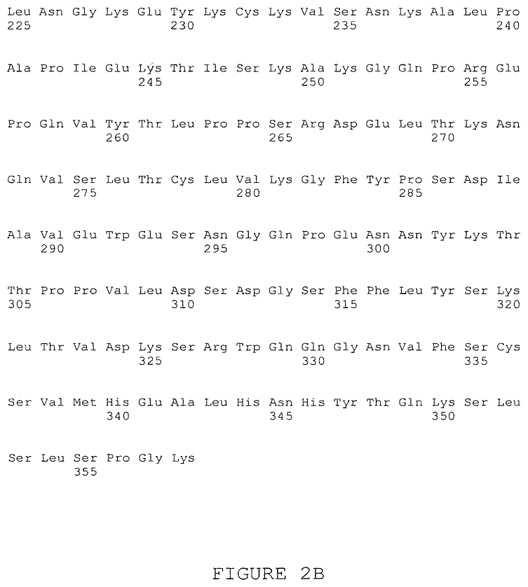

Particular fusions of the present invention include an Flt-1 Ig-like domain 2 with a sequence as represented at positions 24-118 of FIGS. 2A-2B, 6, 8 and 12, which corresponds to amino acids 132-226 of FIG. 10A-10E, or a portion or variant of the sequence that retains the ability to modulate VEGF. In some embodiments, the fusion proteins also bind to placental growth factor.

A signal sequence may also be present and linked to the N-terminus of the soluble protein (e.g., Flt-1 Ig-like domain 2 sequence). The signal sequence may include all of a portion of the native signal sequence, such as all or part of the sequence found at positions 1-26 of FIGS. 10A-10E. In the fusions shown in FIGS. 2A-2B (SEQ ID NO:11), 6 (SEQ ID NO: 15), 8 (SEQ ID NO:17) and 12 (SEQ ID NO:21), a signal sequence of 23 amino acids (amino acids 1-23 of FIGS. 2A-2B, 6, 8 and 12) is present. This sequence is homologous to the native signal sequence of the Flt-1 protein. Alternatively, a heterologous signal sequence can be present. Numerous such sequences are known in the art and will find use herein. Non-limiting examples of signal peptides include those present in secreted proteins such as human growth hormone, bovine growth hormone, bovine proalbumin, human proinsulin, human interferon-γ, human α-fibrinogen, human IgG heavy chain, rat amylase, murine α-fetoprotein, chicken lysozyme and Zea mays rein protein 22.1, brain derived neurotrophic factor, insulin growth factor 1 and β-glucoronidase.

As explained above, the Flt-1 portion of the fusion is linked to a multimerization domain either directly or via a linker moiety. A multimerization domain may be an immunoglobulin sequence, such as an immunoglobulin constant region, a leucine zipper, a hydrophobic region, a hydrophilic region, a polypeptide comprising a free thiol which forms an intermolecular disulfide bond between two or more multimerization domains or, for example a “protuberance-into-cavity” domain described in, for example, U.S. Pat. No. 5,731,168, incorporated herein by reference in its entirety. The multimerization domain provides that portion of the molecule which promotes or allows the formation of dimers, trimers, and the like from monomeric domains.

Multimerization domains will cause at least 5%, 10%, 20%, 30%, 40%, 50%, 60%, 75%, 80%, 85%, 90%, or 95% of the monomeric fusion proteins to migrate on a non-denaturing polyacrylamide gel at a rate appropriate for a multimer. Glycosylation can affect the migration of a protein in a gel. Although particular sequences are shown here, variants such as allelic variants can be used as well. Typically such variants will have at least 85%, 90%, 95%, 97%, 98%, or 99% identity with the disclosed sequence.

Multimerization can be assayed, for example, using reducing and non-reducing gels. Multimerization can also be assayed by detection of increased binding affinity of a protein for its ligand/receptor. BiaCore™ surface plasmon resonance assays can be used in this regard. These assays detect changes in mass by measuring changes in refractive index in an aqueous layer close to a sensor chip surface. Any method known in the art can be used to detect multimerization.

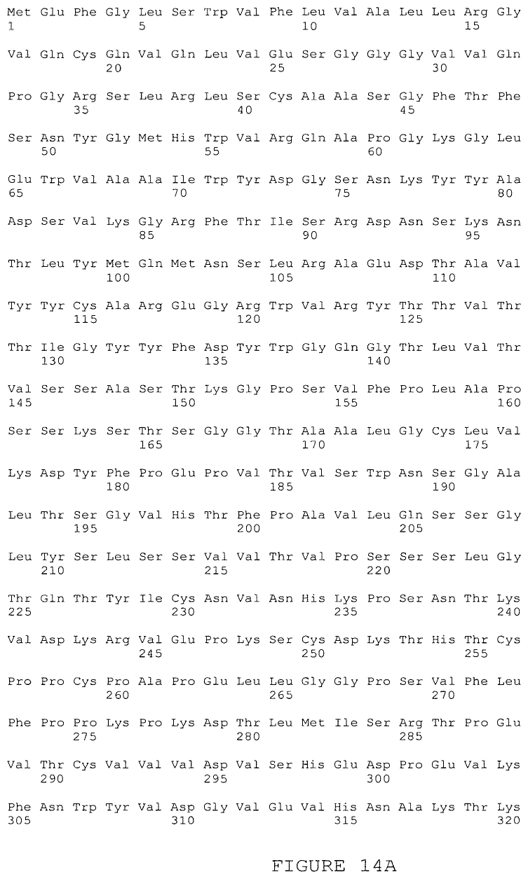

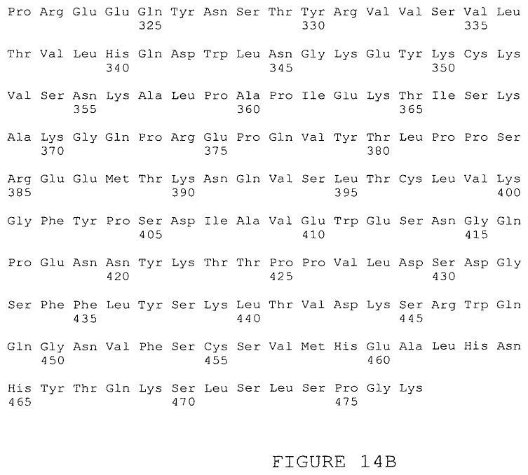

In aspects, multimerization domains are derived from immunoglobulin molecules, including but not limited to regions from the heavy chain, immunoglobulin constant region domains, Fc regions, and the like. Sequences of the Fc portion of IgG1 or IgG2 lambda heavy chain can be used, for example, CH3 alone, such as amino acids 371-477 of FIG. 14A-14B, or portions or extensions of CH3, or both of CH2 and CH3 domains, such as amino acids 247-477 of FIG. 14A-14B, or portions or extensions thereof.

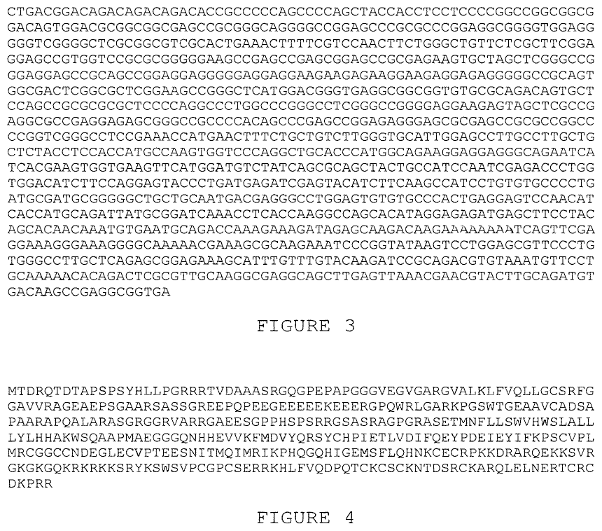

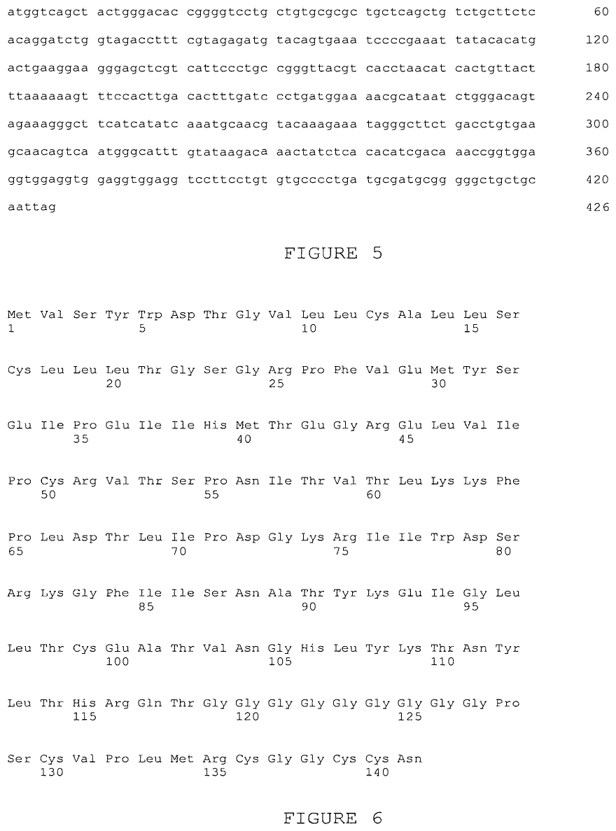

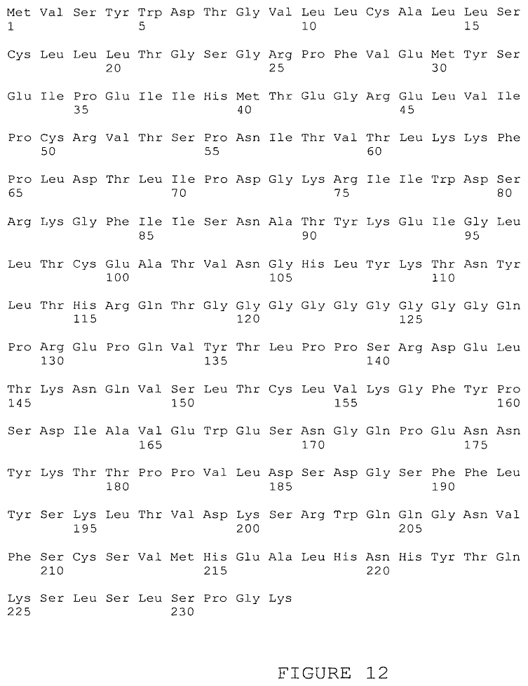

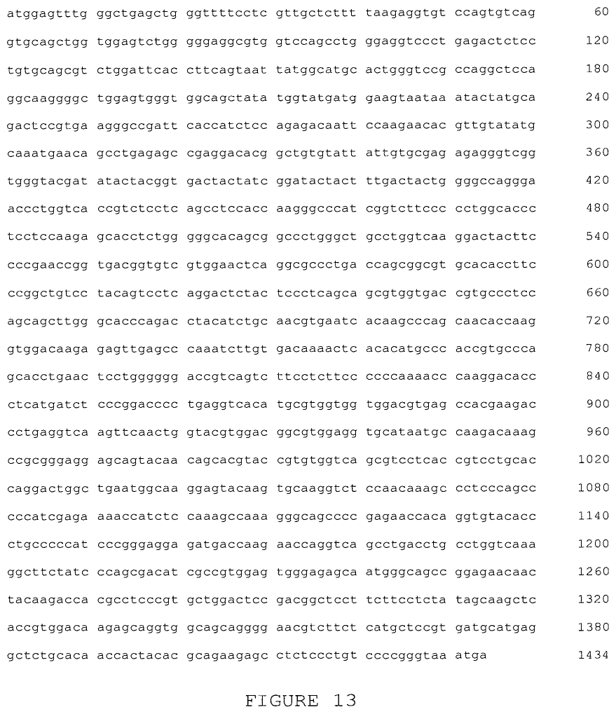

Methods for obtaining portions of immunoglobulin molecule are well known in the art. For example, the Fc portion of an immunoglobulin molecule can be obtained by cleavage of whole antibody molecules with the enzyme papain. Other means can also be used to obtain these portions. For the IgG1 lambda heavy chain protein sequence, see, e.g, Genbank accession no Y14737 and FIGS. 13 (SEQ ID NO:22) and 14A-14B (SEQ ID NO:23), showing the DNA and amino acid sequence, respectively. Other Fc regions can be used, for example, from other IgG types and from IgA, IgM, IgD, or IgE antibodies. The multimerization region of VEGF can also be used. A DNA sequence encoding VEGF is shown at Genbank accession no. NM003376 and FIG. 3 (SEQ ID NO:12). An amino acid sequence of VEGF is shown at Genbank accession no. CAC19513 and FIG. 4 (SEQ ID NO: 13). The multimerization region of VEGF, encoded by VEGF exon 3 (VEGF Ex3), is at about amino acid residues 75-88 of VEGF protein (FIG. 4) and includes the amino acid sequence Pro-Ser-Cys-Val-Pro-Leu-Met-Arg-Cys-Gly-Gly-Cys-Cys-Asn (SEQ ID NO:7).

Although many different linker moieties may be used and may be functionally equivalent, in aspects, a linker of 9 glycine residues is employed in the present invention. Other linkers can be comprised of for example 5-100 amino acid residues, 5-75 amino acid residues, 5-50 amino acid residues, 5-25 amino acid residues, 5-20 amino acid residues, 5-15 amino acid residues, 5-10 amino acid residues, or 5-9 amino acid residues. Examples of useful linkers include:

(SEQ ID NO: 1)
gly₉;
(SEQ ID NO: 2)
glu₉;
(SEQ ID NO: 3)
ser₉;
(SEQ ID NO: 4)
gly₅cyspro₂cys;
(SEQ ID NO: 5)
(gly₄ser)₃;
(SEQ ID NO: 6)
SerCysValProLeuMetArgCysGlyGlyCysCysAsn;
(SEQ ID NO: 7)
ProSerCysValProLeuMetArgCysGlyGlyCysCysAsn;
(SEQ ID NO: 8)
GlyAspLeuIleTyrArgAsnGlnLys;
and
(SEQ ID NO: 9)
Gly₉ProSerCysValProLeuMetArgCysGlyGlyCysCysAsn.

Other polypeptide linkers which can be used include a polyglycine of different lengths, including of 5, 7, or 30 residues. Additionally, other portions of Flt-1 can be used as a linker, for example domain 3 of Flt-1 or portions or extensions thereof, such as amino acids 235-336 of FIGS. 10A-10E.

Linker moieties can also be made from other polymers, such as polyethylene glycol. Such linkers can have from 10 to 1000, 10-500, 10-250, 10-100, or 10-50 ethylene glycol monomer units. Suitable polymers should be of a size similar to the size occupied by the appropriate range of amino acid residues. A typical sized polymer would provide a spacing of from about 10-25 angstroms.

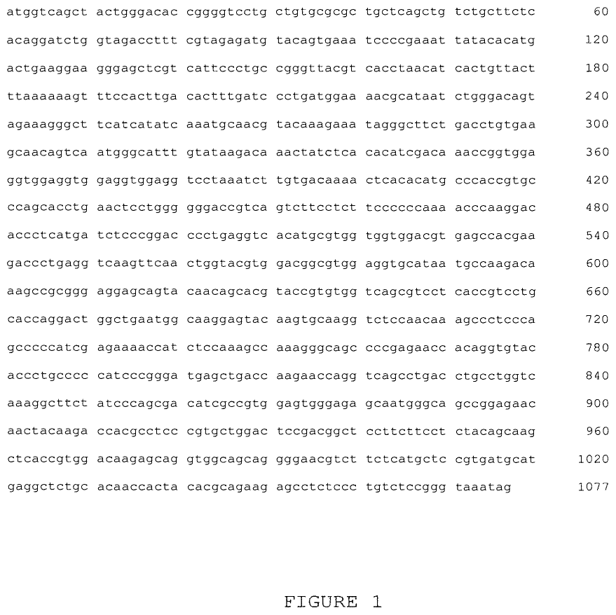

Exemplary forms of the fusion protein used in the invention are shown in FIGS. 2A-2B (SEQ ID NO:11), 6 (SEQ ID NO:15), 8 (SEQ ID NO:17) and 12 (SEQ ID NO:21), encoded by the polynucleotide sequences shown in FIGS. 1 (SEQ ID NO:10), 5 (SEQ ID NO: 14), 7 (SEQ ID NO:16) and 11 (SEQ ID NO:20), respectively. Such sequences are described in U.S. Pat. No. 7,928,072, incorporated herein by reference in its entirety.

The fusion shown in FIGS. 2A-2B (SEQ ID NO:11), termed “sFLT01 protein” herein, includes in N-terminus to C-terminus order, a signal sequence found at positions 1-23 of FIGS. 2A-2B; an Flt-1 Ig-like domain 2 plus extensions of this domain, found at positions 24-118 of FIGS. 2A-2B (corresponding to amino acids 132-226 of FIGS. 10A-10E); a sequence of nine glycines, found at positions 119-127 of FIGS. 2A-2B; and IgG1-Fc CH2/CH3 residues at positions 128-358 of FIGS. 2A-2B.

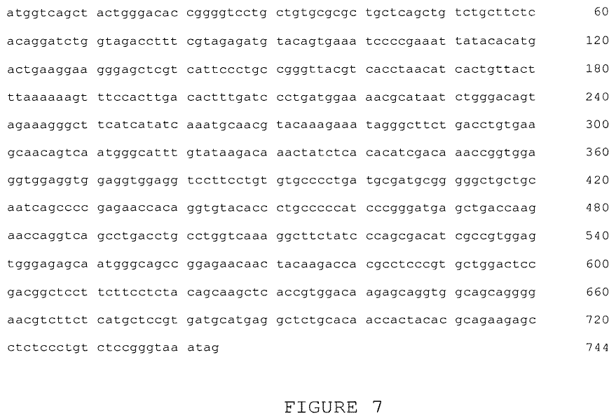

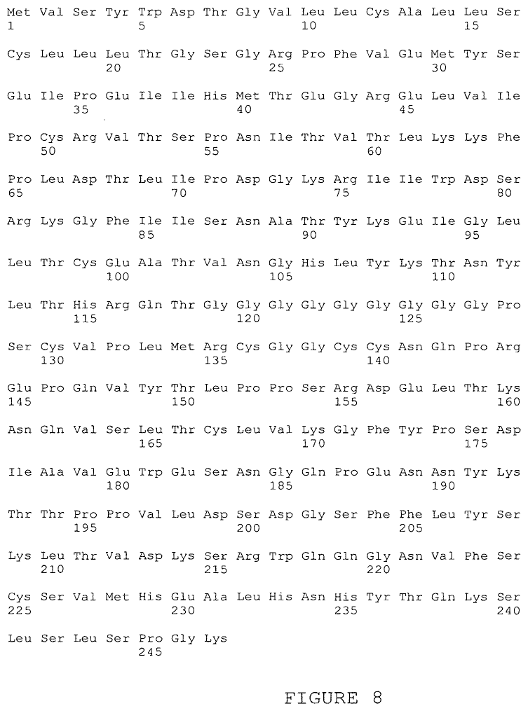

The fusion shown in FIG. 6 (SEQ ID NO:15) includes in N-terminus to C-terminus order, a signal sequence found at positions 1-23 of FIG. 6; an Flt-1 Ig-like domain 2 plus extensions of this domain, found at positions 24-118 of FIG. 6 (corresponding to amino acids 132-226 of FIGS. 10A-10E); a sequence of nine glycines, found at positions 119-127 of FIG. 6; and the VEGF multimerization domain at positions 128-141 of FIG. 6.

FIG. 8 (SEQ ID NO:17) includes in N-terminus to C-terminus order, a signal sequence found at positions 1-23 of FIG. 8; an Flt-1 Ig-like domain 2 plus extensions of this domain, found at positions 24-118 of FIG. 8 (corresponding to amino acids 132-226 of FIGS. 10A-10E); a sequence of nine glycines, found at positions 119-127 of FIG. 8; the VEGF multimerization domain at positions 128-141 of FIG. 8; and a sequence from the IgG CH2/CH3 region at positions 142-247 of FIG. 8.

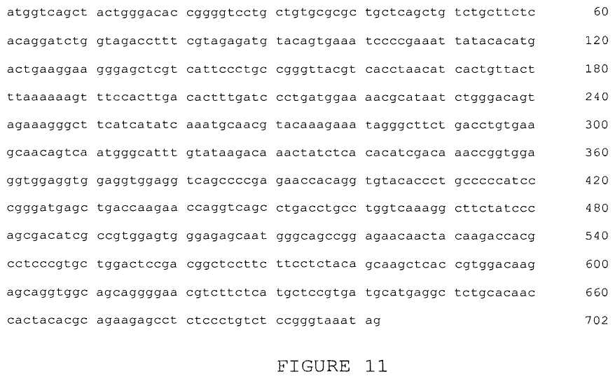

FIG. 12 (SEQ ID NO:21) shows the fusion termed “sFLT02” herein which includes in N-terminus to C-terminus order, a signal sequence found at positions 1-23 of FIG. 12; an Flt-1 Ig-like domain 2 plus extensions of this domain, found at positions 24-118 of FIG. 12 (corresponding to amino acids 132-226 of FIGS. 10A-10E); a sequence of nine glycines, found at positions 119-127 of FIG. 12; and IgG CH2/CH3 residues found at positions 128-233 of FIG. 12.

Although particular sequences are discussed here, variants such as allelic variants can be used as well. Typically such variants will have at least 85%, 90%, 95%, 97%, 98%, or 99% identity with the disclosed sequence and retain the functions described herein, including multimerization and the ability to bind VEFG.

Polynucleotides encoding the Flt-1 receptors and fusions thereof for use with the present invention can be made using standard techniques of molecular biology. For example, polynucleotide sequences coding for the above-described molecules can be obtained using recombinant methods, such as by screening cDNA and genomic libraries from cells expressing the gene, or by deriving the gene from a vector known to include the same. The gene of interest can also be produced synthetically, rather than cloned, based on the known sequences. The molecules can be designed with appropriate codons for the particular sequence. The complete sequence is then assembled from overlapping oligonucleotides prepared by standard methods and assembled into a complete coding sequence. See, e.g., Edge, Nature (1981) 292:756; Nambair et al., Science (1984) 223:1299; and Jay et al., J. Biol. Chem. (1984) 259:6311.

Thus, particular nucleotide sequences can be obtained from vectors harboring the desired sequences or synthesized completely or in part using various oligonucleotide synthesis techniques known in the art, such as site-directed mutagenesis and polymerase chain reaction (PCR) techniques where appropriate. See, e.g., Sambrook, supra. One method of obtaining nucleotide sequences encoding the desired sequences is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al., Proc. Natl. Acad. Sci. USA (1991) 88:4084-4088. Additionally, oligonucleotide-directed synthesis (Jones et al., Nature (1986) 54:75-82), oligonucleotide directed mutagenesis of preexisting nucleotide regions (Riechmann et al., Nature (1988) 332:323-327 and Verhoeyen et al., Science (1988) 239:1534-1536), and enzymatic filling-in of gapped oligonucleotides using T₄ DNA polymerase (Queen et al., Proc. Natl. Acad. Sci. USA (1989) 86:10029-10033) can be used to provide molecules for use in the subject methods.

Once obtained, the polynucleotide encoding the receptor can be linked to a multimerization domain either directly or via a linker moiety, as described above. The constructs can be delivered to a subject using recombinant viral vectors as described further below.

### Gene Delivery Techniques

The sFlt-1 constructs, such as those described above, can be delivered to the subject in question using any of several gene-delivery techniques. Several methods for gene delivery are known in the art. Generally, recombinant vectors are formulated into pharmaceutical compositions as described below and introduced into the subject using either in vivo or ex vivo transduction techniques. If transduced ex vivo, the desired recipient cell will be removed from the subject, transduced with the recombinant vector and reintroduced into the subject. Alternatively, syngeneic or xenogeneic cells can be used where those cells will not generate an inappropriate immune response in the subject.

Suitable methods for the delivery and introduction of transduced cells into a subject have been described. For example, cells can be transduced in vitro by combining recombinant vectors with the subject's cells e.g., in appropriate media, and screening for those cells harboring the DNA of interest using conventional techniques such as Southern blots and/or PCR, or by using selectable markers.

A number of viral based systems have been developed for gene transfer into mammalian cells either in vivo or ex vivo. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems have been described. See, e.g., U.S. Pat. No. 5,219,740; Miller and Rosman, BioTechniques (1989) 7:980-990; Miller, A. D., Human Gene Therapy (1990) 1:5-14; Scarpa et al., Virology (1991) 180:849-852; Burns et al., Proc. Natl. Acad. Sci. USA (1993) 90:8033-8037; and Boris-Lawrie and Temin, Cur. Opin. Genet. Develop. (1993) 3:102-109. Replication-defective murine retroviral vectors are widely utilized gene transfer vectors. Murine leukemia retroviruses include a single strand RNA complexed with a nuclear core protein and polymerase (pol) enzymes encased by a protein core (gag) and surrounded by a glycoprotein envelope (env) that determines host range. The genomic structure of retroviruses include gag, pol, and env genes enclosed at the 5′ and 3′ long terminal repeats (LTRs). Retroviral vector systems exploit the fact that a minimal vector containing the 5′ and 3′ LTRs and the packaging signal are sufficient to allow vector packaging and infection and integration into target cells provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Adenovirus vectors for use in the subject methods are described in more detail below.

Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol, and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875. AAV vector systems are also described in further detail below.

Additional viral vectors which will find use for delivering the nucleic acid molecules of interest include those derived from the pox family of viruses, including vaccinia virus and avian poxvirus. By way of example, vaccinia virus recombinants expressing the genes can be constructed as follows. The DNA encoding the particular polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the protein into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the genes. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as but not limited to vectors derived from the Sindbis and Semliki Forest viruses, will also find use as viral vectors for delivering the polynucleotide encoding the fusion. For a description of Sinbus-virus derived vectors useful for the practice of the instant methods, see, Dubensky et al., J Virol. (1996) 70:508-519; and International Publication Nos. WO 95/07995 and WO 96/17072.

Alternatively, the Flt-1 constructs can be delivered without the use of viral vectors, such as by using plasmid-based nucleic acid delivery systems as described in U.S. Pat. Nos. 6,413,942; 6,214,804; 5,580,859; 5,589,466; 5,763,270; and 5,693,622, all incorporated herein by reference in their entireties. Plasmids will include the gene of interest operably linked to control elements that direct the expression of the protein product in vivo. Such control elements are well known in the art.

### Adenovirus Gene Delivery Systems

In one embodiment of the subject invention, a nucleotide sequence encoding the Flt-1 receptor, such as the fusions described above, is inserted into an adenovirus-based expression vector. The adenovirus genome is a linear double-stranded DNA molecule of approximately 36,000 base pairs with the 55-kDa terminal protein covalently bound to the 5′ terminus of each strand. Adenoviral (“Ad”) DNA contains identical Inverted Terminal Repeats (“ITRs”) of about 100 base pairs with the exact length depending on the serotype. The viral origins of replication are located within the ITRs exactly at the genome ends. DNA synthesis occurs in two stages. First, replication proceeds by strand displacement, generating a daughter duplex molecule and a parental displaced strand. The displaced strand is single-stranded and can form a “panhandle” intermediate, which allows replication initiation and generation of a daughter duplex molecule. Alternatively, replication can proceed from both ends of the genome simultaneously, obviating the requirement to form the panhandle structure.

During the productive infection cycle, the viral genes are expressed in two phases: the early phase, which is the period up to viral DNA replication, and the late phase, which coincides with the initiation of viral DNA replication. During the early phase only the early gene products, encoded by regions E1, E2, E3 and E4, are expressed, which carry out a number of functions that prepare the cell for synthesis of viral structural proteins. During the late phase, late viral gene products are expressed in addition to the early gene products and host cell DNA and protein synthesis are shut off. Consequently, the cell becomes dedicated to the production of viral DNA and of viral structural proteins.

The E1 region of adenovirus is the first region expressed after infection of the target cell. This region consists of two transcriptional units, the E1A and E1B genes. The main functions of the ELA gene products are to induce quiescent cells to enter the cell cycle and resume cellular DNA synthesis, and to transcriptionally activate the E1B gene and the other early regions (E2, E3, E4). Transfection of primary cells with the ELA gene alone can induce unlimited proliferation (immortalization), but does not result in complete transformation. However, expression of E1A in most cases results in induction of programmed cell death (apoptosis), and only occasionally immortalization. Coexpression of the E1B gene is required to prevent induction of apoptosis and for complete morphological transformation to occur. In established immortal cell lines, high level expression of E1A can cause complete transformation in the absence of E1B.

The E1B-encoded proteins assist E1A in redirecting the cellular functions to allow viral replication. The E1B 55 kD and E4 33 kD proteins, which form a complex that is essentially localized in the nucleus, function in inhibiting the synthesis of host proteins and in facilitating the expression of viral genes. Their main influence is to establish selective transport of viral mRNAs from the nucleus to the cytoplasm, concomitantly with the onset of the late phase of infection. The E1B 21 kD protein is important for correct temporal control of the productive infection cycle, thereby preventing premature death of the host cell before the virus life cycle has been completed.

Adenoviral-based vectors express gene product peptides at high levels. Adenoviral vectors have high efficiencies of infectivity, even with low titers of virus. Additionally, the virus is fully infective as a cell-free virion so injection of producer cell lines is not necessary. Adenoviral vectors achieve long-term expression of heterologous genes in vivo. Adenovirus is not associated with severe human pathology, the virus can infect a wide variety of cells and has a broad host-range, the virus can be produced in large quantities with relative ease, and the virus can be rendered replication defective by deletions in the early-region 1 (“E1”) of the viral genome. Thus, vectors derived from human adenoviruses, in which at least the E1 region has been deleted and replaced by a gene of interest, have been used extensively for gene therapy experiments in the pre-clinical and clinical phase.

Adenoviral vectors for use with the present invention are derived from any of the various adenoviral serotypes, including, without limitation, any of the over 40 serotype strains of adenovirus, such as serotypes 2, 5, 12, 40, and 41. The adenoviral vectors used herein are replication-deficient and contain the gene of interest under the control of a suitable promoter, such as any of the promoters discussed below with reference to adeno-associated virus. For example, U.S. Pat. No. 6,048,551, incorporated herein by reference in its entirety, describes replication-deficient adenoviral vectors that include the human gene for the anti-inflammatory cytokine IL-10, as well as vectors that include the gene for the anti-inflammatory cytokine IL-1ra, under the control of the Rous Sarcoma Virus (RSV) promoter, termed Ad.RSVIL-10 and Ad.RSVIL-1ra, respectively.

Other recombinant adenoviruses, derived from any of the adenoviral serotypes, and with different promoter systems, can be used by those skilled in the art. For example, U.S. Pat. No. 6,306,652, incorporated herein by reference in its entirety, describes adenovirus vectors with E2A sequences, containing the hr mutation and the ts125 mutation, termed ts400, to prevent cell death by E2A overexpression, as well as vectors with E2A sequences, containing only the hr mutation, under the control of an inducible promoter, and vectors with E2A sequences, containing the hr mutation and the ts125 mutation (ts400), under the control of an inducible promoter.

Moreover, “minimal” adenovirus vectors as described in U.S. Pat. No. 6,306,652 will find use with the present invention. Such vectors retain at least a portion of the viral genome that is required for encapsidation of the genome into virus particles (the encapsidation signal), as well as at least one copy of at least a functional part or a derivative of the ITR. Packaging of the minimal adenovirus vector can be achieved by co-infection with a helper virus or, alternatively, with a packaging-deficient replicating helper system as described in U.S. Pat. No. 6,306,652.

Other useful adenovirus-based vectors for delivery of the gene of interest include the “gutless” (helper-dependent) adenovirus in which the vast majority of the viral genome has been removed (Wu et al., Anesthes. (2001) 94:1119-1132). Such “gutless” adenoviral vectors essentially create no viral proteins, thus allowing virally driven gene therapy to successfully ensue for over a year after a single administration (Parks, R. J., Clin. Genet. (2000) 58:1-11; Tsai et al., Curr. Opin. Mol. Ther. (2000) 2:515-523) and eliminates interference by the immune system. In addition, removal of the viral genome creates space for insertion of control sequences that provide expression regulation by systemically administered drugs (Burcin et al., Proc. Natl. Acad. Sci. USA (1999) 96:355-360), adding both safety and control of virally driven protein expression. These and other recombinant adenoviruses will find use with the present methods.

### Adeno-Associated Virus Gene Delivery Systems

Adeno-associated virus (AAV) has been used with success to deliver genes for gene therapy. The AAV genome is a linear, single-stranded DNA molecule containing about 4681 nucleotides. The AAV genome generally comprises an internal, nonrepeating genome flanked on each end by inverted terminal repeats (ITRs). The ITRs are approximately 145 base pairs (bp) in length. The ITRs have multiple functions, including providing origins of DNA replication, and packaging signals for the viral genome. The internal nonrepeated portion of the genome includes two large open reading frames, known as the AAV replication (rep) and capsid (cap) genes. The rep and cap genes code for viral proteins that allow the virus to replicate and package into a virion. In particular, a family of at least four viral proteins are expressed from the AAV rep region, Rep 78, Rep 68, Rep 52, and Rep 40, named according to their apparent molecular weight. The AAV cap region encodes at least three proteins, VP1, VP2, and VP3.

AAV has been engineered to deliver genes of interest by deleting the internal nonrepeating portion of the AAV genome (i.e., the rep and cap genes) and inserting a heterologous gene (in this case, the gene encoding the Flt-1 receptor or fusion) between the ITRs. The heterologous gene is typically functionally linked to a heterologous promoter (constitutive, cell-specific, or inducible) capable of driving gene expression in the patient's target cells under appropriate conditions. Termination signals, such as polyadenylation sites, can also be included.

AAV is a helper-dependent virus; that is, it requires coinfection with a helper virus (e.g., adenovirus, herpesvirus or vaccinia), in order to form AAV virions. In the absence of coinfection with a helper virus, AAV establishes a latent state in which the viral genome inserts into a host cell chromosome, but infectious virions are not produced. Subsequent infection by a helper virus “rescues” the integrated genome, allowing it to replicate and package its genome into an infectious AAV virion. While AAV can infect cells from different species, the helper virus must be of the same species as the host cell. Thus, for example, human AAV will replicate in canine cells coinfected with a canine adenovirus.

Recombinant AAV virions comprising the gene of interest may be produced using a variety of art-recognized techniques described more fully below. Wild-type AAV and helper viruses may be used to provide the necessary replicative functions for producing rAAV virions (see, e.g., U.S. Pat. No. 5,139,941, incorporated herein by reference in its entirety). Alternatively, a plasmid, containing helper function genes, in combination with infection by one of the well-known helper viruses can be used as the source of replicative functions (see e.g., U.S. Pat. Nos. 5,622,856 and 5,139,941, both incorporated herein by reference in their entireties). Similarly, a plasmid, containing accessory function genes can be used in combination with infection by wild-type AAV, to provide the necessary replicative functions. These three approaches, when used in combination with a rAAV vector, are each sufficient to produce rAAV virions. Other approaches, well known in the art, can also be employed by the skilled artisan to produce rAAV virions.

In one embodiment of the present invention, a triple transfection method (described in detail in U.S. Pat. No. 6,001,650, incorporated by reference herein in its entirety) is used to produce rAAV virions because this method does not require the use of an infectious helper virus, enabling rAAV virions to be produced without any detectable helper virus present. This is accomplished by use of three vectors for rAAV virion production: an AAV helper function vector, an accessory function vector, and a rAAV expression vector. One of skill in the art will appreciate, however, that the nucleic acid sequences encoded by these vectors can be provided on two or more vectors in various combinations.

As explained herein, the AAV helper function vector encodes the “AAV helper function” sequences (i.e., rep and cap), which function in trans for productive AAV replication and encapsidation. The AAV helper function vector can support efficient AAV vector production without generating any detectable wt AAV virions (i.e., AAV virions containing functional rep and cap genes). An example of such a vector, pHLP19, is described in U.S. Pat. No. 6,001,650, incorporated herein by reference in its entirety. The rep and cap genes of the AAV helper function vector can be derived from any of the known AAV serotypes, as explained above. For example, the AAV helper function vector may have a rep gene derived from AAV-2 and a cap gene derived from AAV-6; one of skill in the art will recognize that other rep and cap gene combinations are possible, the defining feature being the ability to support rAAV virion production.

The accessory function vector encodes nucleotide sequences for non-AAV-derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the well-known helper viruses such as adenovirus, herpesvirus (other than herpes simplex virus type-1), and vaccinia virus. In embodiments, the accessory function plasmid pLadeno5 is used (details regarding pLadeno5 are described in U.S. Pat. No. 6,004,797, incorporated herein by reference in its entirety). This plasmid provides a complete set of adenovirus accessory functions for AAV vector production, but lacks the components necessary to form replication-competent adenovirus.

In order to further an understanding of AAV, a more detailed discussion is provided below regarding recombinant AAV expression vectors and AAV helper and accessory functions.

### Recombinant AAV Expression Vectors

Recombinant AAV (rAAV) expression vectors are constructed using known techniques to at least provide as operatively linked components in the direction of transcription, control elements including a transcriptional initiation region, the polynucleotide of interest and a transcriptional termination region. The control elements are selected to be functional in the cell of interest, such as in a mammalian cell. The resulting construct which contains the operatively linked components is bounded (5′ and 3′) with functional AAV ITR sequences.

The nucleotide sequences of AAV ITR regions are known. See, e.g., Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.) for the AAV-2 sequence. AAV ITRs used in the vectors of the invention need not have a wild-type nucleotide sequence, and may be altered, e.g., by the insertion, deletion or substitution of nucleotides. Additionally, AAV ITRs may be derived from any of several AAV serotypes, including without limitation, AAV1, AAV2, AAV3, AAV4, AAV5, AAV6, AAV7, AAV8, AAV9, AAVrh8, AAVrh8R, AAV10, AAVrh10, AAV11, AAV12, and the like. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

Suitable polynucleotide molecules for use in AAV vectors will be less than about 5 kilobases (kb) in size. The selected polynucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, neuron-specific enolase promoter, a GFAP promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, CA).

The AAV expression vector which harbors the polynucleotide molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al. (1988) Molec. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol, and Immunol. 158:97-129; Kotin (1994) Human Gene Therapy 5:793-801; Shelling and Smith (1994) Gene Therapy 1:165-169; and Zhou et al. (1994) J. Exp. Med. 179:1867-1875.

Alternatively, AAV ITRs can be excised from the viral genome or from an AAV vector containing the same and fused 5′ and 3′ of a selected nucleic acid construct that is present in another vector using standard ligation techniques, such as those described in Sambrook et al., supra. For example, ligations can be accomplished in 20 mM Tris-Cl pH 7.5, 10 mM MgCl2, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration). AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226.

For the purposes of the invention, suitable host cells for producing rAAV virions from the AAV expression vectors include microorganisms, yeast cells, insect cells, and mammalian cells, that can be, or have been, used as recipients of a heterologous DNA molecule and that are capable of growth in, for example, suspension culture, a bioreactor, or the like. The term includes the progeny of the original cell which has been transfected. Thus, a “host cell” as used herein generally refers to a cell which has been transfected with an exogenous DNA sequence. Cells from the stable human cell line, 293 (readily available through, e.g., the American Type Culture Collection under Accession Number ATCC CRL1573) can be used in the practice of the present invention. Particularly, the human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

### AAV Helper Functions

Host cells containing the above-described AAV expression vectors must be rendered capable of providing AAV helper functions in order to replicate and encapsidate the nucleotide sequences flanked by the AAV ITRs to produce rAAV virions. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs, namely the rep and cap coding regions, or functional homologues thereof.

By “AAV rep coding region” is meant the art-recognized region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. For a description of the AAV rep coding region, see, e.g., Muzyczka, N. (1992) Current Topics in Microbiol, and Immunol. 158:97-129; and Kotin, R. M. (1994) Human Gene Therapy 5:793-801. Suitable homologues of the AAV rep coding region include the human herpesvirus 6 (HHV-6) rep gene which is also known to mediate AAV-2 DNA replication (Thomson et al. (1994) Virology 204:304-311).

By “AAV cap coding region” is meant the art-recognized region of the AAV genome which encodes the capsid proteins VP1, VP2, and VP3, or functional homologues thereof. These Cap expression products supply the packaging functions which are collectively required for packaging the viral genome. For a description of the AAV cap coding region, see, e.g., Muzyczka, N. and Kotin, R. M. (supra).

AAV helper functions are introduced into the host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection. AAV helper constructs lack AAV ITRs and can neither replicate nor package themselves.

These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al. (1989) J. Virol. 63:3822-3828; and McCarty et al. (1991) J. Virol. 65:2936-2945. A number of other vectors have been described which encode Rep and/or Cap expression products. See, e.g., U.S. Pat. No. 5,139,941.

### AAV Accessory Functions

The host cell (or packaging cell) must also be rendered capable of providing nonAAV-derived functions, or “accessory functions,” in order to produce rAAV virions. Accessory functions are nonAAV-derived viral and/or cellular functions upon which AAV is dependent for its replication. Thus, accessory functions include at least those nonAAV proteins and RNAs that are required in AAV replication, including those involved in activation of AAV gene transcription, stage specific AAV mRNA splicing, AAV DNA replication, synthesis of Cap expression products and AAV capsid assembly. Viral-based accessory functions can be derived from any of the known helper viruses.

In particular, accessory functions can be introduced into and then expressed in host cells using methods known to those of skill in the art. Typically, accessory functions are provided by infection of the host cells with an unrelated helper virus. A number of suitable helper viruses are known, including adenoviruses; herpesviruses such as herpes simplex virus types 1 and 2; and vaccinia viruses. Nonviral accessory functions will also find use herein, such as those provided by cell synchronization using any of various known agents. See, e.g., Buller et al. (1981) J. Virol. 40:241-247; McPherson et al. (1985) Virology 147:217-222; Schlehofer et al. (1986) Virology 152:110-117.

Alternatively, accessory functions can be provided using an accessory function vector as defined above. See, e.g., U.S. Pat. No. 6,004,797 and International Publication No. WO 01/83797, incorporated herein by reference in their entireties. Nucleic acid sequences providing the accessory functions can be obtained from natural sources, such as from the genome of an adenovirus particle, or constructed using recombinant or synthetic methods known in the art. As explained above, it has been demonstrated that the full-complement of adenovirus genes are not required for accessory helper functions. In particular, adenovirus mutants incapable of DNA replication and late gene synthesis have been shown to be permissive for AAV replication. Ito et al., (1970) J. Gen. Virol. 9:243; Ishibashi et al, (1971) Virology 45:317. Similarly, mutants within the E2B and E3 regions have been shown to support AAV replication, indicating that the E2B and E3 regions are probably not involved in providing accessory functions. Carter et al., (1983) Virology 126:505. However, adenoviruses defective in the E1 region, or having a deleted E4 region, are unable to support AAV replication. Thus, E1A and E4 regions are likely required for AAV replication, either directly or indirectly. Laughlin et al., (1982) J. Virol. 41:868; Janik et al., (1981) Proc. Natl. Acad. Sci. USA 78:1925; Carter et al., (1983) Virology 126:505. Other characterized Ad mutants include: E1B (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)). Although studies of the accessory functions provided by adenoviruses having mutations in the E1B coding region have produced conflicting results, Samulski et al., (1988) J. Virol. 62:206-210, has reported that E1B55k is required for AAV virion production, while E1B 19k is not. In addition, International Publication WO 97/17458 and Matshushita et al., (1998) Gene Therapy 5:938-945, describe accessory function vectors encoding various Ad genes. Accessory function vectors can comprise an adenovirus VA RNA coding region, an adenovirus E4 ORF6 coding region, an adenovirus E2A 72 kD coding region, an adenovirus E1A coding region, and an adenovirus E1B region lacking an intact E1B55k coding region. Such vectors are described in International Publication No. WO 01/83797.

As a consequence of the infection of the host cell with a helper virus, or transfection of the host cell with an accessory function vector, accessory functions are expressed which transactivate the AAV helper construct to produce AAV Rep and/or Cap proteins. The Rep expression products excise the recombinant DNA (including the DNA of interest) from the AAV expression vector. The Rep proteins also serve to duplicate the AAV genome. The expressed Cap proteins assemble into capsids, and the recombinant AAV genome is packaged into the capsids. Thus, productive AAV replication ensues, and the DNA is packaged into rAAV virions. A “recombinant AAV virion,” or “rAAV virion” is defined herein as an infectious, replication-defective virus including an AAV protein shell, encapsidating a heterologous nucleotide sequence of interest which is flanked on both sides by AAV ITRs.

Following recombinant AAV replication, rAAV virions can be purified from the host cell using a variety of conventional purification methods, such as column chromatography, CsCl gradients, and the like. For example, a plurality of column purification steps can be used, such as purification over an anion exchange column, an affinity column and/or a cation exchange column. See, for example, International Publication No. WO 02/12455. Further, if infection is employed to express the accessory functions, residual helper virus can be inactivated, using known methods. For example, adenovirus can be inactivated by heating to temperatures of approximately 60° C. for, e.g., 20 minutes or more. This treatment effectively inactivates only the helper virus since AAV is extremely heat stable while the helper adenovirus is heat labile.

The resulting rAAV virions containing the nucleotide sequence of interest can then be used for gene delivery using the techniques described below.

rAAV Particles

In some embodiments, the viral particle is a recombinant AAV particle comprising a nucleic acid comprising a transgene flanked by one or two ITRs. The nucleic acid is encapsidated in the AAV particle. The AAV particle also comprises capsid proteins. In some embodiments, the nucleic acid comprises the protein coding sequence(s) of interest (e.g., a therapeutic transgene) operatively linked components in the direction of transcription, control sequences including transcription initiation and termination sequences, thereby forming an expression cassette. The expression cassette is flanked on the 5′ and 3′ end by at least one functional AAV ITR sequences. By “functional AAV ITR sequences” it is meant that the ITR sequences function as intended for the rescue, replication and packaging of the AAV virion. See Davidson et al., PNAS, 2000, 97 (7) 3428-32; Passini et al., J. Virol., 2003, 77 (12): 7034-40; and Pechan et al., Gene Ther., 2009, 16:10-16, all of which are incorporated herein in their entirety by reference. For practicing some aspects of the invention, the recombinant vectors comprise at least all of the sequences of AAV essential for encapsidation and the physical structures for infection by the rAAV. AAV ITRs for use in the vectors of the invention need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99 (18): 11854-6; Gao et al., PNAS, 2003, 100 (10): 6081-6; and Bossis et al., J. Virol., 2003, 77 (12): 6799-810. Use of any AAV serotype is considered within the scope of the present invention. In some embodiments, a rAAV vector is a vector derived from an AAV serotype, including without limitation, AAV1, AAV2, AAV3, AAV4, AAV5, AA6, AAV7, AAV8, AAV9, AAVrh.8, AAVrh. 10, AAV11, AAV12, or the like. In some embodiments, the nucleic acid in the AAV comprises an ITR of AAV1, AAV2, AAV3, AAV4, AAV5, AA6, AAV7, AAV8, AAV9, AAVrh.8, AAVrh10, AAV11, AAV12 or the like. In further embodiments, the rAAV particle comprises capsid proteins of AAV1, AAV2, AAV3, AAV4, AAV5, AA6, AAV7, AAV8, AAV9, AAVrh.8, AAVrh. 10, AAV11, AAV12 or the like. In further embodiments, the rAAV particle comprises capsid proteins of an AAV serotype from Clades A-F (Gao, et al. J. Virol. 2004, 78 (12): 6381).

Different AAV serotypes are used to optimize transduction of particular target cells or to target specific cell types within a particular target tissue (e.g., a diseased tissue). A rAAV particle can comprise viral proteins and viral nucleic acids of the same serotype or a mixed serotype. Any combination of AAV serotypes for production of a rAAV particle is provided herein as if each combination had been expressly stated herein.

### Self-Complementary AAV Viral Genomes

In some aspects, the invention provides viral particles comprising a recombinant self-complementing genome. AAV viral particles with self-complementing genomes and methods of use of self-complementing AAV genomes are described in U.S. Pat. Nos. 6,596,535; 7,125,717; 7,765,583; 7,785,888; 7,790,154; 7,846,729; 8,093,054; and 8,361,457; and Wang Z., et al., (2003) Gene Ther 10:2105-2111, each of which are incorporated herein by reference in its entirety. A rAAV comprising a self-complementing genome will quickly form a double stranded DNA molecule by virtue of its partially complementing sequences (e.g., complementing coding and non-coding strands of a transgene). In some embodiments, the invention provides an AAV viral particle comprising an AAV genome, wherein the rAAV genome comprises a first heterologous polynucleotide sequence (e.g., a therapeutic transgene coding strand) and a second heterologous polynucleotide sequence (e.g., the noncoding or antisense strand of the therapeutic transgene) wherein the first heterologous polynucleotide sequence can form intrastrand base pairs with the second polynucleotide sequence along most or all of its length. In some embodiments, the first heterologous polynucleotide sequence and a second heterologous polynucleotide sequence are linked by a sequence that facilitates intrastrand basepairing; e.g., a hairpin DNA structure. Hairpin structures are known in the art, for example in siRNA molecules. In some embodiments, the first heterologous polynucleotide sequence and a second heterologous polynucleotide sequence are linked by a mutated ITR (e.g., the right ITR). The mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

Production of rAAV Vectors

Numerous methods are known in the art for production of rAAV vectors, including transfection, stable cell line production, and infectious hybrid virus production systems which include adenovirus-AAV hybrids, herpesvirus-AAV hybrids and baculovirus-AAV hybrids. rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a transgene (such as a therapeutic transgene) flanked by at least one AAV ITR sequences; and 5) suitable media and media components to support rAAV production. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors.

Suitable rAAV production culture media of the present invention may be supplemented with serum or serum-derived recombinant proteins at a level of 0.5%-20% (v/v or w/v). Alternatively, as is known in the art, rAAV vectors may be produced in serum-free conditions which may also be referred to as media with no animal-derived products. One of ordinary skill in the art may appreciate that commercial or custom media designed to support production of rAAV vectors may also be supplemented with one or more cell culture components known in the art, including without limitation glucose, vitamins, amino acids, and/or growth factors, in order to increase the titer of rAAV in production cultures.

rAAV production cultures can be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

rAAV vector particles of the invention may be harvested from rAAV production cultures by lysis of the host cells of the production culture or by harvest of the spent media from the production culture, provided the cells are cultured under conditions known in the art to cause release of rAAV particles into the media from intact cells, as described more fully in U.S. Pat. No. 6,566,118. Suitable methods of lysing cells are also known in the art and include for example multiple freeze/thaw cycles, sonication, microfluidization, and treatment with chemicals, such as detergents and/or proteases.

Purification of rAAV Vectors

At harvest, rAAV production cultures of the present invention may contain one or more of the following: (1) host cell proteins; (2) host cell DNA; (3) plasmid DNA; (4) helper virus; (5) helper virus proteins; (6) helper virus DNA; and (7) media components including, for example, serum proteins, amino acids, transferrins and other low molecular weight proteins. In addition, rAAV production cultures further include rAAV particles having an AAV capsid serotype selected from the group consisting of AAV1, AAV2, AAV3, AAV4, AAV5, AAV6, AAV7, AAV8, AAVrh8, AAV9, AAV10, AAVrh10, AAV11, AAV12, or the like.

Thus, in some embodiments, the rAAV production culture harvest is clarified to remove host cell debris. In some embodiments, the production culture harvest is clarified by filtration through a series of depth filters including, for example, a grade DOHC Millipore Millistak⁺ HC Pod Filter, a grade A1HC Millipore Millistak⁺ HC Pod Filter, and a 0.2 μm Filter Opticap XL10 Millipore Express SHC Hydrophilic Membrane filter. Clarification can also be achieved by a variety of other standard techniques known in the art, such as, centrifugation or filtration through any cellulose acetate filter of 0.2 μm or greater pore size known in the art.

In some embodiments, the rAAV production culture harvest is further treated with Benzonase® to digest any high molecular weight DNA present in the production culture. In some embodiments, the Benzonase® digestion is performed under standard conditions known in the art including, for example, a final concentration of 1-2.5 units/ml of Benzonase® at a temperature ranging from ambient to 37° C. for a period of 30 minutes to several hours.

rAAV particles may be isolated or purified using one or more of the following purification steps: centrifugation, flow-through anionic exchange filtration, tangential flow filtration (TFF) for concentrating the rAAV particles, rAAV capture by apatite chromatography, heat inactivation of helper virus, rAAV capture by hydrophobic interaction chromatography, buffer exchange by size exclusion chromatography (SEC), nanofiltration, and rAAV capture by anionic exchange chromatography. These steps may be used alone, in various combinations, or in different orders. In some embodiments, the method comprises all the steps in the order as described below. Methods to purify rAAV particles are found, for example, in U.S. Pat. Nos. 6,989,264 and 8,137,948 and WO 2010/148143.

### Compositions and Delivery

Once produced, the sFlt-1 receptor, or vectors (or virions) encoding the same, such as the fusions described above, will be formulated into compositions suitable for direct delivery to the eye in order to treat macular degeneration. If gene therapy is desired, compositions will comprise sufficient genetic material to produce a therapeutically effective amount of the Flt-1 of interest, e.g., an amount sufficient to bind to and mediate the effects of the corresponding signal pathway, or to reduce or ameliorate symptoms of the disease state in question, or an amount sufficient to confer the desired benefit. Appropriate doses will also depend on the condition of the subject being treated, age, the severity of the condition being treated, the mode of administration, among other factors. An appropriate effective amount can be readily determined by one of skill in the art.

Thus, a “therapeutically effective amount” will fall in a relatively broad range that can be determined through clinical trials. For example, for in vivo injection of rAAV virions, a therapeutically effective dose will be on the order of from about 10⁶ to 10¹⁵ vector genomes (vg) of the recombinant virus, such as 10⁸ to 10¹⁴ vg, for example 10⁸ to 10¹² vg, such as 10⁸ to 10¹⁰ vg, 10⁸ to 10⁹ vg, or any integer in between, such as 0.5×10⁸ vg . . . 1×10⁸ vg . . . 1.5×10⁸ vg . . . 2×10⁸ vg . . . 5×10⁸ vg . . . 1×10⁹ vg . . . 2×10⁹ vg . . . 3×10⁹ vg . . . 5×10⁹ vg . . . 6×10⁹ vg . . . 1×10¹⁰ vg . . . 2×10¹⁰ vg . . . 5×10¹⁰ vg . . . 1×10¹¹ vg . . . 5×10¹¹ vg . . . 1×10¹² vg . . . 5×10¹² vg, etc.

In aspects, the compositions will also contain ophthalmologically acceptable excipients. The compositions can be formulated as solutions, gels, ointments, suspensions, a dry powder to be reconstituted with a vehicle before use, or as other suitable and well-tolerated ophthalmic delivery systems. Such excipients include any pharmaceutical agent suitable for direct delivery to the eye which may be administered without undue toxicity. Pharmaceutically acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

Administration can be effected in one dose, continuously or intermittently throughout the course of treatment. Methods of determining the most effective means of administration are well known to those of skill in the art and will vary with the vector, the composition of the therapy, the target cells, and the subject being treated. Single and multiple administrations can be carried out with the dose level and pattern being selected by the treating physician.

If multiple doses are administered, the first formulation administered can be the same or different than the subsequent formulations. Thus, for example, the first administration can be in the form of an AAV virion and the second administration in the form of an adenovirus vector, plasmid DNA, an AAV virion, a subunit vaccine composition, or the like. Moreover, subsequent delivery can also be the same or different than the second mode of delivery.

It should be understood that more than one transgene can be expressed by the delivered recombinant vector. Alternatively, separate vectors, each expressing one or more different transgenes, can also be delivered to the subject as described herein. Thus, multiple transgenes can be delivered concurrently or sequentially. Furthermore, it is also intended that the vectors delivered by the methods of the present invention be combined with other suitable compositions and therapies. For instance, other compounds for treating macular degeneration can be present.

As explained above, for delivery of the sFlt-1 receptor constructs to the eye (whether via gene therapy or protein therapy), administration will typically be local. This has the advantage of limiting the amount of material (protein or DNA) that needs to be administered and limiting systemic side-effects. Many possible modes of delivery can be used, including, but not limited to: topical administration on the cornea by a gene gun; subconjunctival injection, intracameral injection, via eye drops to the cornea, injection into the anterior chamber via the temporal limbus, intrastromal injection, corneal application combined with electrical pulses, intracorneal injection, subretinal injection, intravitreal injection (e.g., front, mid or back vitreal injection), and intraocular injection. Alternatively cells can be transfected or transduced ex vivo and delivered by intraocular implantation. See, Auricchio, Mol. Ther. (2002) 6:490-494; Bennett, Nature Med. (1996) 2:649-654; Borras, Experimental Eye Research (2003) 76:643-652; Chaum, Survey of Ophthalmology (2002) 47:449-469; Campochiaro, Expert Opinions in Biological Therapy (2002) 2:537-544; Lai, Gene Therapy (2002) 9:804-813; Pleyer, Progress in Retinal and Eye Research (2003) 22:277-293.

Thus, the ophthalmic formulations are administered in any form suitable for ocular drug administration, e.g., dosage forms suitable for topical administration, a solution or suspension for administration as eye drops, eye washes, or injection, ointment, gel, liposomal dispersion, colloidal microparticle suspension, or the like, or in an ocular insert, e.g., in an optionally biodegradable controlled release polymeric matrix. The ocular insert is implanted in the conjunctiva, sclera, pars plana, anterior segment, or posterior segment of the eye. Implants provide for controlled release of the formulation to the ocular surface, typically sustained release over an extended time period. Additionally, in embodiments, the formulation is entirely composed of components that are naturally occurring and/or as GRAS (“Generally Regarded as Safe”) by the U.S. Food and Drug Administration.

Combinations of protein and nucleic acid treatments can be used. For example, a fusion protein according to the invention can be administered to a patient. If a favorable response is observed, then a nucleic acid molecule encoding the fusion protein can be administered for a long term effect. Alternatively, the protein and nucleic acid can be administered simultaneously or approximately simultaneously.

Dosage treatment may be a single dose schedule or a multiple dose schedule. Moreover, the subject may be administered as many doses as appropriate. One of skill in the art can readily determine an appropriate number of doses.

In aspects, the compositions described herein are used in any of the methods described herein.

### Kits of the Invention

The invention also provides kits. In certain embodiments, the kits of the invention comprise one or more containers comprising a purified sFlt-1 receptor, fusions comprising the same, recombinant vectors encoding the same, or AAV virions/rAAV vectors encoding the same. In embodiments, the kits contain an ophthalmologically acceptable excipients. The kits can also comprise delivery devices suitable for ocular delivery. The kits may further comprise a suitable set of instructions, generally written instructions, relating to the use of the kit and its contents for any of the methods described herein.

The kits may comprise the components in any convenient, appropriate packaging. For example, if the nucleic acid, protein, vector, or virion are provided as a dry formulation (e.g., freeze dried or a dry powder), a vial with a resilient stopper can be used, so that the vectors may be resuspended by injecting fluid through the resilient stopper. Ampules with non-resilient, removable closures (e.g., sealed glass) or resilient stoppers can be used for liquid formulations. Also contemplated are packages for use in combination with a specific device (e.g., a syringe).

The instructions generally include information as to dosage, dosing schedule, and route of administration for the intended method of use. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also contemplated.

### 2. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Materials and Methods

### Soluble Vector Construction

FIGS. 1 (SEQ ID NO:10) and 2A-2B (SEQ ID NO:11) show the DNA and protein sequences of the fusion protein termed “sFLT01”. This construct includes in N-terminus to C-terminus order, a signal sequence found at positions 1-23 of FIGS. 2A-2B; an Flt-1 Ig-like domain 2 plus extensions of this domain, found at positions 24-118 of FIGS. 2A-2B; a sequence of nine glycines, found at positions 119-127 of FIGS. 2A-2B; and IgG1-Fc CH2/CH3 residues at positions 128-358 of FIGS. 2A-2B.

DNA was cloned into plasmid pCBA(2)-int-BGH, which contains a hybrid chicken β-actin (CBA) promoter and a bovine growth hormone polyadenylation signal sequence (BGH poly A). Xu et al., Hum. Gene. Ther. (2001) 12:563-573.

The whole sFLT01 expression cassette was then cloned into a previral plasmid vector pAAVSP70 containing AAV2 inverted terminal repeats (ITRs). Ziegler et al, Mol. Ther. (2004) 9:231-240. The total size of the resulting AAV genome in plasmid sp70.BR/sFLT01 including the region flanked by the ITRs was 4.6 kb.

The recombinant vector AAV2-sFLT01 was produced by triple transfection of 293 cells using helper plasmids p5rep-Δ-CMVcap and pHelper (Stratagene, La Jolla, CA, USA), and purified according to the protocol using an iodixanol step gradient and a HiTrap Heparin column (GE Healthcare Life Sciences, Piscataway, NJ, USA) on an AKTA FPLC system (GE Healthcare Life Sciences, Piscataway, NJ). Vincent et al, J. Virol. (1997) 71:1897-1905; Zolotukhin et al., Methods (2002) 28:158-167.

Viral titers were determined using a real-time TaqMan PCR assay (ABI Prism 7700; Applied Biosystems, Foster City, CA, USA) with primers that were specific for the BGH poly A sequence.

### Intravitreal Injection.

For example 1, female cynomolgus monkeys (Macaca fascicularis) 2.1-2.8 kg were sedated with ketamine and diazepam. Prior to dose administration, the eye was cleaned with a povidone-iodine topical antiseptic and rinsed with sterile saline. A mydriatic (1% tropicamide) and a topical anesthetic (proparacaine) were instilled into each injected eye. A lid speculum was inserted to keep the lids open during the procedure and the globe was retracted. The 27 gauge needle of the dose syringe was inserted through the sclera and pars plana approximately 4 mm posterior to the limbus. The needle was directed posterior to the lens into one of three locations: the anterior vitreous adjacent to the peripheral retina, the mid-vitreous or the posterior vitreous adjacent to the macula. The AAV vector was injected in a total volume of 50 μl or 100 μl.

### Induction of Choroidal Neovascularization (CNV).

CNV was induced in the primates after the administration of the test article to allow sufficient time for the transgene to reach peak expression. A diode laser with a 532 nm wavelength (Iridex Corp., Mountain View, CA) and a slit lamp adapter was used to rupture Bruch's membrane to induce CNV. Nine burns were placed on the macular region in a 3×3 grid pattern using the same type laser operated with a spot size of 75 microns at 500-700 mW for 100-200 milliseconds.

### CNV Evaluation.

Leakage from the CNV lesions in monkeys was evaluated 2, 3 and 4 weeks following laser induction by fluorescein angiography. Sedated animals were injected with a fluorescein dye (10% fluorescein sodium, approximately 0.1 mL/kg) and the fundus was imaged at several time points following dye injection to monitor the arterial and venous phases. Funduscopic images were collected and analyzed for the presence of leaking CNV at each burn site.

### Example 1

### Efficacy of AAV2-sFLT01 in Non-Human Primates

Two studies were conducted in non-human primates (NHP) to determine the efficacy of intravitreally administered AAV2-sFLT01. In the first study (Study A), cynomolgus monkeys were treated intravitreally with 2×10⁸ or 2×10⁹ vg of AAV2-sFLT01. The contralateral control eye was treated with the same dose of an AAV2 vector that did not code for a transgene (AAV2-Null). Laser CNV induction occurred 6 weeks following vector administration. The degree of CNV was found to be maximal at the 3 week fluorescein angiography, therefore this was the time point used to evaluate the efficacy of treatment. The number of leaking lesions was compared between the AAV2-sFLT01 treated and the contralateral control eye (Table 1). None of the sFLT01 treatment groups demonstrated a statistically significant reduction in leaking CNV lesions compared to the AAV2-Null control eyes.

In the second study (Study B), 2×10¹⁰ vg of AAV2-sFLT01 or AAV2-Null was delivered intravitreally while the contralateral eyes were kept naive to treatment. Laser CNV induction in both eyes occurred 22 weeks following vector administration. All six of the AAV2-sFLT01 treated eyes demonstrated a significant reduction in the amount of CNV leakage compared to the naive contralateral control eyes with only 7% of the AAV2-sFLT01 treated burns exhibiting leaking CNV while 56% of the burns in the control eye were leaking. This difference was statistically significant (p<0.0001) as determined by Fisher's exact test. Eyes treated with the AAV2-Null control vector did not demonstrate a reduction in CNV compared to the untreated control eyes.

TABLE 1
Results from two NHP efficacy studies.

| | | | Average | | | |
|---|---|---|---|---|---|---|
| | Dose (vg)/ | Laser Induction | sFLT01 | Percentage of | | |
| | Injection | (weeks post | Expression | Leaking Lesions | | Number of |
| Study | Placement | administration) | (ng/mL) | Treated Eye | Control Eye | Animals |
| A | 2 × 10⁸ | 6 | 26 | 28% | 50% | 2 |
| | 2 × 10⁹ | 6 | 190 | 67% | 67% | 4 |
| B | 2 × 10¹⁰ | 22 | 1,833 | 7% | 56% | 6 |
| | 2 × 10¹⁰/ | 22 | n/a | 63% | 48% | 3 |
| | Central VH | | | | | |
| | Null Vector | | | | | |

The ipsilateral eye from Study A received AAV2-sFLT01 vector while the contralateral control eye received AAV2-Null vector six weeks prior to laser induction of CNV. In Study B, the ipsilateral eye received AAV2-sFLT01 vector while the contralateral eye remained naive to treatment six weeks prior to laser induction of CNV in both eyes. The average sFLT01 expression level at the time of laser induction is presented in the table.

In sum, intravitreal administration of an AAV2 gene therapy vector encoding for a soluble receptor to VEGF resulted in transduction of retinal cells with dose dependent expression of the transgene product in the non-human primate eye. Expression was first measured as early as three weeks following administration and was found to be relatively stable to the last time point measured (23 weeks).

Efficacy was observed in the NHP model for seven out of eight animals whose sFLT01 expression levels were above 100 ng/mL in aqueous humor suggesting that there may be a threshold value of sFLT01 that must be achieved to effect a change in neovascularization in this model. All six of the animals treated with 2×10¹⁰ vg that were lasered 22 weeks following vector administration had reduced CNV compared to the control eyes.

### Example 2

### Efficacy of AAV2-sFLT01 in Humans

Dose escalation studies were conducted in humans to evaluate the safety, tolerability and efficacy of a single intravitreal injection of AAV2-sFLT01. AAV2-sFLT01 was produced as described above. Patients used in the study were end-stage neovascular AMD patients. Criteria for qualifying for the study included the following:
Choroidal neovascular membrane (CNV) secondary to AMD, as confirmed by the patient's medical history and a documented diagnosis of CNV.
Distance best corrected visual acuity (BCVA) of 20/100 or worse in the study eye.
The fellow eye must have distance BCVA of 20/400 or better.
The study eye, i.e., the eye that received AAV2-sFLT01, had the worst CVA (as compared to the fellow eye).
Subfoveal disciform scarring in the study eye for the dose-escalation part of the study. Patients may or may not have macular scarring in the study eye for the second part of the study (maximum tolerated dose (MTD) phase). In addition, patients enrolled in the second part of the study must have demonstrated responsiveness to an anti-VEGF therapy within 12 months prior to screening and after the patient's most recent treatment of anti-VEGF therapy.
Noted presence of intra- or subretinal fluid.
Adequate dilation of pupils to permit thorough ocular examination and testing.
   Exclusion Criteria were as Follows:
CNV in the study eye due to any reason other than AMD.
History of conditions in the study eye during Screening which might alter visual acuity or interfere with study testing.
Active uncontrolled glaucoma.
Had any intraocular surgeries in the study eye within 3 months of enrollment or are known or likely candidates for intraocular surgery (including cataract surgery) in the study eye within 1 year of treatment.
Acute or chronic infection in the study eye.
History of inflammation in the study eye or ongoing inflammation in either eye.
Any contraindication to intravitreal injection.
Received Photo Dynamic Therapy in the study eye within 60 days, or laser photocoagulation within 14 days prior to Screening.
Currently using or have used ranibizumab (Lucentis®), bevacizumab (Avastin™), or pegaptanib sodium (Macugen®) within 1 month prior to Screening.
Currently using or have used Aflibercept (Eylea®) within 4 months prior to Screening.
Currently using any periocular (study eye), intravitreal (study eye), or systemic (oral or intravenous) steroids within 3 months prior to Screening.
Any active herpetic infection, in particular active lesions in the eye or on the face.
Any significant poorly controlled illness that would preclude study compliance and follow-up.
Current or prior use of any medication known to be toxic to the retina or optic nerve.
Previous treatment with any ocular or systemic gene transfer product.
Received any investigational product within 120 days prior to Screening.

In the first part of the study, four separate groups of patients were administered a fixed volume of 100 μL of different doses of AAV2-sFLT01 as follows. (1) Group 1 received a single intravitreal injection in one eye of 2×10⁸ vg; (2) Group 2 received a single intravitreal injection in one eye of 2×10⁹ vg; (3) Group 3 received a single intravitreal injection in one eye of 6×10⁹ vg; (4) Group 4 received a single intravitreal injection in one eye of 2×10¹⁰ vg.

These doses were determined to be safe and well tolerated. In particular, no dose-limiting toxicity (DLT) was observed and MTD was not reached.

In order to determine the efficacy of AAV2-sFLT01, changes from baseline in the amount of subretinal and intraretinal fluid was measured by optical coherence tomography (OCT). Additionally BCVA was measured as were sFLT01 protein levels in the aqueous fluid via anterior chamber taps.

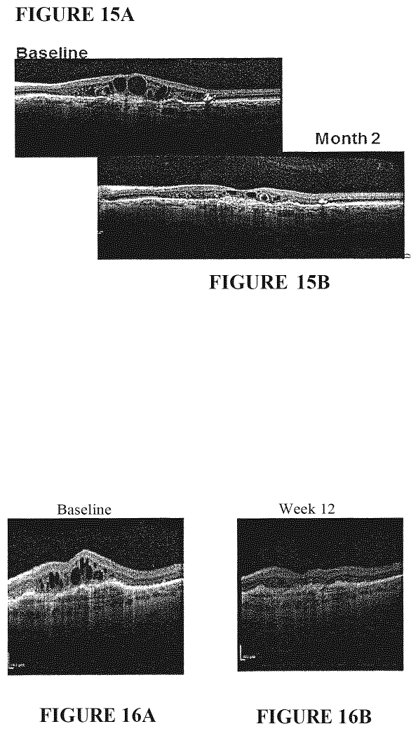

Surprisingly, a patient that received a single intravitreal injection of 2×10⁸ vg displayed a significant reduction of subretinal and intraretinal fluid as measured by OCT. See, FIGS. 15A and 15B.

In the second part of the study, a single intravitreal injection of the highest dose used in the first study (2×10¹⁰ vg) was given to different patients. This dose also resulted in a significant reduction of subretinal and intraretinal fluid as measured by OCT two months after injection. See, FIGS. 16A and 16B.

Table 2 shows the number of expected responders and non-responders. An expected responder was characterized as a patient that was expected to show a response to anti-VEGF treatments based upon their baseline characteristics. Expected responders were then characterized as follows: Full responders: Patients that showed robust response, dry retina, and return of normal retinal anatomy with no additional treatments needed. Partial responder: Patients that showed some decrease of fluid. Non-responder: No effect seen.

TABLE 2
Biological Activity

| | EXPECTED | EXPECTED NON- |
|---|---|---|
| N = 19^{a} | RESPONDER (N = 11) | RESPONDER (N = 7) |
| Responder | 4^{b} | 0 |
| Partial Responder | 2 | 0 |
| Non Responder | 5 | 7 |

^{a}One patient was unassessable.
^{b}Among four total responders: one out three years, one out two years, one out one year and one out 18 weeks.

As shown in Table 2, six of eleven expected responders showed at least a partial response to treatment.

Thus, methods for treating macular degeneration, as well as compositions comprising sFlt-1 receptors and fusions thereof, are described. Although embodiments of the subject invention have been described in some detail, it is understood that obvious variations can be made without departing from the spirit and the scope of the invention as defined herein.

1. A method of treating macular degeneration in a human subject comprising administering to the diseased eye of the subject a composition comprising a recombinant adeno-associated virus (rAA V) virion comprising a polynucleotide encoding a soluble protein comprising at least one domain of VEGFR-1 (Flt-1) capable of modulating VEGF activity, wherein from about 1×10⁷ to about 1×10¹³ rAAV virions are delivered to the eye.

2. The method of claim 1, wherein the method comprises reducing intraocular pressure, retinal thickness, subretinal fluids, or intraretinal fluids.

3. A method of treating macular edema in a human subject comprising administering to the diseased eye of the subject a composition comprising a recombinant adeno-associated virus (rAAV) virion comprising a polynucleotide encoding a soluble protein comprising at least one domain of VEGFR-1 (Flt-1) capable of modulating VEGF activity, wherein from about 1×10⁷ to about 1×10¹³ rAAV virions are delivered to the eye.

4. The method of claim 3, wherein the method comprises reducing intraocular pressure, retinal thickness, subretinal fluids, or intraretinal fluids.

5. The method of any one of claims 1-4, wherein from about 1×10⁷ to about 1×10¹²; 1×10⁸ to about 1×10¹²; about 1×10⁸ to about 1×10¹¹; about 1×10⁸ to about 1×10¹⁰; about 1×10⁶ to about 1×10⁹; about 2×10⁷ to about 2×10¹²; about 2×10⁸ to about 2×10¹²; about 2×10⁸ to about 2×10¹¹; about 2×10⁸ to about 2×10¹⁰; about 2×10⁴ to about 2×10⁹; 2×10⁹ to about 2×10¹⁰; about 1×10¹⁰ to about 1×10¹³; about 1×10¹⁰ to about 1×10¹²; about 1×10¹⁰ to about 1×10¹¹; about 2×10¹⁰ to about 1×10¹³; 2×10¹⁰ to about 1×10¹²; about 2×10¹⁰ to about 2×10¹²; about 2×10¹⁰ to about 1×10¹¹; or about 2×10¹⁰ to about 2×10¹¹ rAAV virions are administered to the eye.

6. The method of any one of claims 1-5, wherein about 1×10⁷, about 2×10⁷, about 6×10⁷, about 1×10⁸, about 2×10⁸, about 6×10⁸, about 1×10⁹, about 2×10⁹, about 6×10⁹, about 1×10¹⁰, about 2×10¹⁰, about 6×10¹⁰, about 1×10¹¹, about 2×10¹¹, about 6×10¹¹, about 1×10¹², about 2×10¹², about 6×10¹², or about 1×10¹³ rAAV virions are administered to the eye.

7. A method of treating macular degeneration in a human subject comprising administering to the diseased eye of the subject a composition comprising a recombinant adeno-associated virus (rAAV) virion comprising a polynucleotide encoding a soluble protein comprising at least one domain of VEGFR-1 (Flt-1) capable of modulating VEGF activity, wherein less than about 2×10¹⁰ rAAV virions are delivered to the eye.

8. The method of claim 7, wherein the method comprises reducing intraocular pressure, retinal thickness, subretinal fluids, or intraretinal fluids.

9. A method of treating macular edema in a human subject comprising administering to the diseased eye of the subject a composition comprising a recombinant adeno-associated virus (rAAV) virion comprising a polynucleotide encoding a soluble protein comprising at least one domain of VEGFR-1 (Flt-1) capable of modulating VEGF activity, wherein less than about 2×10¹⁰ rAAV virions are delivered to the eye.

10. The method of claim 9, wherein the method comprises reducing intraocular pressure, retinal thickness, subretinal fluids, or intraretinal fluids.

11. The method of any one of claims 7-10, wherein from about 2×10⁸ to less than 2×10¹⁰ rAAV virions are delivered to the eye.

12. The method of any one of claims 7-10, wherein up to about 2×10⁸ rAAV virions are delivered to the eye.

13. The method of any one of claims 7-10, wherein up to about 2×10⁹ rAAV virions are delivered to the eye.

14. The method of any one of claims 1-13, wherein the composition further comprises an ophthalmologically acceptable vehicle.

15. The method of any one of claims 1-14, wherein a single intravitreal injection of rAAV virions is administered to the eye.

16. The method of any one of claims 1-15, wherein the soluble protein comprises:
(a) the at least one domain of Flt-1;
(b) a multimerization domain derived from an immunoglobulin heavy chain; and
(c) a linker 5-25 amino acid residues in length linking (a) to (b),
wherein when the soluble protein is expressed, a multimer of the soluble protein is produced.

17. The method of any one of claims 1-16, wherein the at least one domain comprises domain 2 of Flt-1.

18. The method of claim 16 or 17, wherein the multimer is a homodimer.

19. The method of any one of claims 16-18, wherein the multimerization domain comprises the Fc region of an IgG, or an active fragment thereof.

20. The method of any one of claims 16-19, wherein the multimerization domain comprises the CH3 domain of an IgG, or an active fragment thereof.

21. The method of any one of claims 16-20, wherein the multimerization domain is from an IgG1, an IgG2, an IgG3 or an IgG4.

22. The method of claim 21, wherein the multimerization domain is from the constant region of an IgG1 heavy chain.

23. The method of any one of claims 16-22 wherein the linker is selected from the group consisting of:
(SEQ ID NO: 1)
gly₉;
(SEQ ID NO: 2)
glu₉;
(SEQ ID NO: 3)
ser₉;
(SEQ ID NO: 4)
gly₅cyspro₂cys;
(SEQ ID NO: 5)
(gly₄ser)₃;
(SEQ ID NO: 6)
SerCysValProLeuMetArgCysGlyGlyCysCysAsn;
(SEQ ID NO: 7)
ProSerCysValProLeuMetArgCysGlyGlyCysCysAsn;
(SEQ ID NO: 8)
GlyAspLeuIleTyrArgAsnGlnLys;
and
(SEQ ID NO: 9)
Gly₉ProSerCysValProLeuMetArgCysGlyGlyCysCysAsn.

24. The method of any one of claims 16-23, wherein the soluble protein has the formula X-Y-Z, wherein X comprises the IgG-like domain 2 of Flt-1, wherein Y is Gly₉, and wherein Z is an IgG Fc region or an IgG CH3 region.

25. The method of any one of claims 16-24, wherein the multimerization domain is humanized.

26. The method of any one of claims 16-25, wherein the soluble protein comprises an amino acid sequence selected from the group consisting of (a) the amino acid sequence depicted in FIGS. 2A-2B (SEQ ID NO:11); (b) the amino acid sequence depicted in FIG. 6 (SEQ ID NO:15); (c) the amino acid sequence depicted in FIG. 8 (SEQ ID NO:17); (d) the amino acid sequence depicted in FIG. 12 (SEQ ID NO:21); and (e) an active variant of (a), (b), (c) or (d) having at least 90% sequence identity thereto.

27. The method of any one of claims 1, 2, 5-8, and 11-26, wherein the macular degeneration is age-related macular degeneration (AMD).

28. The method of claim 27, wherein the macular degeneration is wet AMD.

29. The method of any one of the preceding claims, wherein the rAAV virion is derived from an AAV serotype selected from AAV1, AAV2, AAV3, AAV4, AAV5, AAV6, AAV7, AAV8, AAV9, AAVrh8, AAVrh8R, AAV10, AAVrh10, AAV11 or AAV12.

30. The method of claim 29, wherein the rAAV virion is derived from AAV2.

31. Use of a recombinant adeno-associated virus (rAAV) virion comprising a polynucleotide encoding a soluble protein comprising at least one domain of VEGFR-1 (Flt-1) capable of modulating VEGF activity, in the manufacture of a composition for treating macular degeneration in a human subject by delivering about 1×10⁷ to about 1×10¹³ rAAV virions to the eye.

32. The use of claim 31, wherein intraocular pressure, retinal thickness, subretinal fluids, or intraretinal fluids are reduced.

33. Use of a recombinant adeno-associated virus (rAAV) virion comprising a polynucleotide encoding a soluble protein comprising at least one domain of VEGFR-1 (Flt-1) capable of modulating VEGF activity in the manufacture of a composition for treating macular edema in a human subject by delivering about 1×10⁷ to about 1×10¹³ rAAV virions to the eye.

34. The use of claim 33, wherein intraocular pressure, retinal thickness, subretinal fluids, or intraretinal fluids are reduced.

35. The use of any one of claims 31-34, wherein from about 1×10⁷ to about 1×10¹²; 1×10⁸ to about 1×10¹²; about 1×10⁸ to about 1×10¹¹; about 1×10⁸ to about 1×10¹⁰; about 1×10⁸ to about 1×10⁹; about 2×10⁷ to about 2×10¹²; about 2×10⁸ to about 2×10¹²; about 2×10⁸ to about 2×10¹¹; about 2×10⁸ to about 2×10¹⁰; about 2×10⁸ to about 2×10⁹; 2×10⁹ to about 2×10¹⁰; about 1×10¹⁰ to about 1×10¹³; about 1×10¹⁰ to about 1×10¹²; about 1×10¹⁰ to about 1×10¹¹; about 2×10¹⁰ to about 1×10¹³; 2×10¹⁰ to about 1×10¹²; about 2×10¹⁰ to about 2×10¹²; about 2×10¹⁰ to about 1×10¹¹; or about 2×10¹⁰ to about 2×10¹¹ rAAV virions are delivered to the eye.

36. The use of any one of claims 31-35, wherein about 1×10⁷ about 2×10⁷, about 6×10⁷, about 1×10⁸, about 2×10⁸, about 6×10⁸, about 1×10⁹, about 2×10⁹, about 6×10⁹, about 1×10¹⁰, about 2×10¹⁰, about 6×10¹⁰, about 1×10¹¹, about 2×10¹¹, about 6×10¹¹, about 1×10¹², about 2×10¹² about 6×10¹² or about 1×10¹³ rAAV virions are delivered to the eye.

37. Use of a recombinant adeno-associated virus (rAAV) virion comprising a polynucleotide encoding a soluble protein comprising at least one domain of VEGFR-1 (Flt-1) capable of modulating VEGF activity in the manufacture of a composition for treating macular degeneration in a human subject by delivering less than about 2×10¹⁰ rAAV virions to the eye.

38. The use of claim 37, wherein intraocular pressure, retinal thickness, subretinal fluids, or intraretinal fluids are reduced.

39. Use of a recombinant adeno-associated virus (rAAV) virion comprising a polynucleotide encoding a soluble protein comprising at least one domain of VEGFR-1 (Flt-1) capable of modulating VEGF activity in the manufacture of a composition for treating macular edema in a human subject by delivering less than about 2×10¹⁰ rAAV virions to the eye.

40. The use of claim 39, wherein intraocular pressure, retinal thickness, subretinal fluids, or intraretinal fluids are reduced.

41. The use of any one of claims 37-40, wherein from about 2×10⁸ to less than 2×10¹⁰ rAAV virions are delivered to the eye.

42. The use ofany one of claims 37-40, wherein up to about 2×10⁸ rAAV virions are delivered to the eye.

43. The use of any one of claims 37-40, wherein up to about 2×10⁹ rAAV virions are delivered to the eye.

44. The use of any one of claims 31-43, wherein the composition further comprises an ophthalmologically acceptable vehicle.

45. The use of any one of claims 31-44, wherein a single intravitreal injection of rAAV virions is delivered to the eye.

46. The use of any one of claims 31-45, wherein the soluble protein comprises:
(a) the at least one domain of Flt-1;
(b) a multimerization domain derived from an immunoglobulin heavy chain; and
(c) a linker 5-25 amino acid residues in length linking (a) to (b),
wherein when the soluble protein is expressed, a multimer of the soluble protein is produced.

47. The use of any one of claims 31-46, wherein the at least one domain comprises domain 2 of Flt-1.

48. The use of claim 46 or 47, wherein the multimer is a homodimer.

49. The use of any one of claims 46-48, wherein the multimerization domain comprises the Fc region of an IgG, or an active fragment thereof.

50. The use of any one of claims 46-49, wherein the multimerization domain comprises the CH3 domain of an IgG, or an active fragment thereof.

51. The use of any one of claims 46-50, wherein the multimerization domain is from an IgG1, an IgG2, an IgG3 or an IgG4.

52. The use of claim 51, wherein the multimerization domain is from the constant region of an IgG1 heavy chain.

53. The use of any one of claims 46-52 wherein the linker is selected from the group consisting of:
(SEQ ID NO: 1)
gly₉;
(SEQ ID NO: 2)
glu₉;
(SEQ ID NO: 3)
ser₉;
(SEQ ID NO: 4)
gly₅cyspro₂cys;
(SEQ ID NO: 5)
(gly₄ser)₃;
(SEQ ID NO: 6)
SerCysValProLeuMetArgCysGlyGlyCysCysAsn;
(SEQ ID NO: 7)
ProSerCysValProLeuMetArgCysGlyGlyCysCysAsn;
(SEQ ID NO: 8)
GlyAspLeuIleTyrArgAsnGlnLys;
and
(SEQ ID NO: 9)
Gly₉ProSerCysValProLeuMetArgCysGlyGlyCysCysAsn.

54. The use of any one of claims 46-53, wherein the soluble protein has the formula X-Y-Z, wherein X comprises the IgG-like domain 2 of Flt-1, wherein Y is Gly₉, and wherein Z is an IgG Fc region or an IgG CH3 region.

55. The use of any one of claims 46-54, wherein the multimerization domain is humanized.

56. The use of any one of claims 46-55, wherein the soluble protein comprises an amino acid sequence selected from the group consisting of (a) the amino acid sequence depicted in FIGS. 2A-2B (SEQ ID NO:11); (b) the amino acid sequence depicted in FIG. 6 (SEQ ID NO:15); (c) the amino acid sequence depicted in FIG. 8 (SEQ ID NO:17); (d) the amino acid sequence depicted in FIG. 12 (SEQ ID NO:21); and (e) an active variant of (a), (b), (c) or (d) having at least 90% sequence identity thereto.

57. The use of any one of claims 31, 32, 35-38, and 41-56, wherein the macular degeneration is age-related macular degeneration (AMD).

58. The use of claim 57, wherein the macular degeneration is wet AMD.

59. The use of any one of claims 31-58, wherein the rAAV virion is derived from an AAV serotype selected from AAV1, AAV2, AAV3, AAV4, AAV5, AAV6, AAV7, AAV8, AAV9, AAVrh8, AAVrh8R, AAV10, AAVrh10, AAV11 or AAV12.

60. The use of claim 59, wherein the rAAV virion is derived from AAV2.


SEQUENCE LISTING (23 sequences)

SEQ ID NO: 1 - protein, 9 aa, synthetic construct
  REGION 1..9: note = Synthetic
GGGGGGGGG

SEQ ID NO: 2 - protein, 9 aa, synthetic construct
  REGION 1..9: note = Synthetic
EEEEEEEEE

SEQ ID NO: 3 - protein, 9 aa, synthetic construct
  REGION 1..9: note = Synthetic
SSSSSSSSS

SEQ ID NO: 4 - protein, 9 aa, synthetic construct
  REGION 1..9: note = Synthetic
GGGGGCPPC

SEQ ID NO: 5 - protein, 15 aa, synthetic construct
  REGION 1..15: note = Synthetic
GGGGSGGGGSGGGGS

SEQ ID NO: 6 - protein, 13 aa, synthetic construct
  REGION 1..13: note = Synthetic
SCVPLMRCGGCCN

SEQ ID NO: 7 - protein, 14 aa, synthetic construct
  REGION 1..14: note = Synthetic
PSCVPLMRCGGCCN

SEQ ID NO: 8 - protein, 9 aa, synthetic construct
  REGION 1..9: note = Synthetic
GDLIYRNQK

SEQ ID NO: 9 - protein, 23 aa, synthetic construct
  REGION 1..23: note = Synthetic
GGGGGGGGGPSCVPLMRCGGCCN

SEQ ID NO: 10 - DNA, 1077 bp, synthetic construct
  misc_feature 1..1077: note = sFLT01 protein: DNA sequence for a fusion protein including Flt-1
  source 1..1077: mol_type = other DNA
atggtcagctactgggacaccggggtcctgctgtgcgcgctgctcagctgtctgcttctcacaggatctggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccggtggaggtggaggtggaggtggaggtcctaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 11 - protein, 358 aa, synthetic construct
  REGION 1..358: note = amino acid sequence for the sFLT01 protein
MVSYWDTGVLLCALLSCLLLTGSGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTGGGGGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1188 bp, Homo sapiens
  misc_feature 1..1188: note = DNA sequence encoding VEGF
  source 1..1188: mol_type = genomic DNA
ctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 13 - protein, 395 aa, Homo sapiens
  REGION 1..395: note = amino acid sequence for VEGF
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 14 - DNA, 426 bp, synthetic construct
  misc_feature 1..426: note = DNA sequence for an additional fusion protein including a soluble Flt-1 linked by a Gly9 linker to the VEGF multimerization domain, Ex3
  source 1..426: mol_type = other DNA
atggtcagctactgggacaccggggtcctgctgtgcgcgctgctcagctgtctgcttctcacaggatctggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccggtggaggtggaggtggaggtggaggtccttcctgtgtgcccctgatgcgatgcgggggctgctgcaattag

SEQ ID NO: 15 - protein, 141 aa, synthetic construct
  REGION 1..141: note = Synthetic amino acid sequence encoded by the DNA sequence of Figure 5
MVSYWDTGVLLCALLSCLLLTGSGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTGGGGGGGGGPSCVPLMRCGGCCN

SEQ ID NO: 16 - DNA, 744 bp, synthetic construct
  misc_feature 1..744: note = DNA sequence for an additional fusion protein including a soluble Flt-1 linked by Gly9 to the VEGF multimerization domain, Ex3 and a sequence from the IgG1 CH3 region
  source 1..744: mol_type = other DNA
atggtcagctactgggacaccggggtcctgctgtgcgcgctgctcagctgtctgcttctcacaggatctggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccggtggaggtggaggtggaggtggaggtccttcctgtgtgcccctgatgcgatgcgggggctgctgcaatcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 17 - protein, 247 aa, synthetic construct
  REGION 1..247: note = Synthetic amino acid sequence encoded by the DNA sequence of Figure 7
MVSYWDTGVLLCALLSCLLLTGSGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTGGGGGGGGGPSCVPLMRCGGCCNQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 7120 bp, Homo sapiens
  misc_feature 1..7120: note = DNA sequence encoding for a representative Flt-1 receptor protein
  source 1..7120: mol_type = genomic DNA
atcgaggtccgcgggaggctcggagcgcgccaggcggacactcctctcggctcctccccggcagcggcggcggctcggagcgggctccggggctcgggtgcagcggccagcgggcgcctggcggcgaggattacccggggaagtggttgtctcctggctggagccgcgagacgggcgctcagggcgcggggccggcggcggcgaacgagaggacggactctggcggccgggtcgttggccgcggggagcgcgggcaccgggcgagcaggccgcgtcgcgctcaccatggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctctttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcctgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatacgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctagagtttgacacgaagccttatttctagaagcacatgtgtatttatacccccaggaaactagcttttgccagtattatgcatatataagtttacacctttatctttccatgggagccagctgctttttgtgatttttttaatagtgcttttttttttttgactaacaagaatgtaactccagatagagaaatagtgacaagtgaagaacactactgctaaatcctcatgttactcagtgttagagaaatccttcctaaacccaatgacttccctgctccaacccccgccacctcagggcacgcaggaccagtttgattgaggagctgcactgatcacccaatgcatcacgtaccccactgggccagccctgcagcccaaaacccagggcaacaagcccgttagccccagggatcactggctggcctgagcaacatctcgggagtcctctagcaggcctaagacatgtgaggaggaaaaggaaaaaaagcaaaaagcaagggagaaaagagaaaccgggagaaggcatgagaaagaatttgagacgcaccatgtgggcacggagggggacggggctcagcaatgccatttcagtggcttcccagctctgacccttctacatttgagggcccagccaggagcagatggacagcgatgaggggacattttctggattctgggaggcaagaaaaggacaaatatcttttttggaactaaagcaaattttagaactttacctatggaagtggttctatgtccattctcattcgtggcatgttttgatttgtagcactgagggtggcactcaactctgagcccatacttttggctcctctagtaagatgcactgaaaacttagccagagttaggttgtctccaggccatgatggccttacactgaaaatgtcacattctattttgggtattaatatatagtccagacacttaactcaatttcttggtattattctgttttgcacagttagttgtgaaagaaagctgagaagaatgaaaatgcagtcctgaggagaggagttttctccatatcaaaacgagggctgatggaggaaaaaggtcaataaggtcaagggaaaaccccgtctctataccaaccaaaccaattcaccaacacagttgggacccaaaacacaggaagtcagtcacgtttccttttcatttaatggggattccactatctcacactaatctgaaaggatgtggaagagcattagctggcgcatattaagcactttaagctccttgagtaaaaaggtggtatgtaatttatgcaaggtatttctccagttgggactcaggatattagttaatgagccatcactagaagaaaagcccattttcaactgctttgaaacttgcctggggtctgagcatgatgggaatagggagacagggtaggaaagggcgcctactcttcagggtctaaagatcaagtgggccttggatcgctaagctggctctgtttgatgctatttatgcaagttagggtctatgtatttatgatgtctgcaccttctgcagccagtcagaagctggagaggcaacagtggattgctgcttcttggggagaagagtatgcttccttttatccatgtaatttaactgtagaacctgagctctaagtaaccgaagaatgtatgcctctgttcttatgtgccacatccttgtttaaaggctctctgtatgaagagatgggaccgtcatcagcacattccctagtgagcctactggctcctggcagcggcttttgtggaagactcactagccagaagagaggagtgggacagtcctctaccaagatctaaatccaaacaaaagcaggctagagccagaagagaggacaaatctttgttcttcctcttctttacatacgcaaaccacctgtgacagctggcaattttataaatcaggtaactggaaggaggttaaacacagaaaaaagaagacctcagtcaattctctacttttttttttttttccaaatcagataatagcccagcaaatagtgataacaaataaaaccttagctattcatgtcttgatttcaataattaattcttaatcattaagagaccataataaatactccttttcaagagaaaagcaaaaccattagaattgttactcagctccttcaaactcaggtttgtagcatacatgagtccatccatcagtcaaagaatggttccatctggagtcttaatgtagaaagaaaaatggagacttgtaataatgagctagttacaaagtgcttgttcattaaaatagcactgaaaattgaaacatgaattaactgataatattccaatcatttgccatttatgacaaaaatggttggcactaacaaagaacgagcacttcctttcagagtttctgagataatgtacgtggaacagtctgggtggaatggggctgaaaccatgtgcaagtctgtgtcttgtcagtccaagaagtgacaccgagatgttaattttagggacccgtgccttgtttcctagcccacaagaatgcaaacatcaaacagatactcgctagcctcatttaaattgattaaaggaggagtgcatctttggccgacagtggtgtaactgtatgtgtgtgtgtgtgtgtgtgtgtgtgtgtgtgtgtgggtgtatgtgtgttttgtgcataactatttaaggaaactggaattttaaagttacttttatacaaaccaagaatatatgctacagatataagacagacatggtttggtcctatatttctagtcatgatgaatgtattttgtataccatcttcatataataaacttccaaaacaca

SEQ ID NO: 19 - protein, 1338 aa, Homo sapiens
  REGION 1..1338: note = amino acid sequence of a representative Flt-1 receptor protein
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLLIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 20 - DNA, 702 bp, synthetic construct
  misc_feature 1..702: note = DNA sequence for a fusion protein including Flt-1, termed "sFLT02 protein" herein which includes a soluble Flt-1 linked by Gly9 to the a sequence from the IgG1 CH3 region
  source 1..702: mol_type = other DNA
atggtcagctactgggacaccggggtcctgctgtgcgcgctgctcagctgtctgcttctcacaggatctggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccggtggaggtggaggtggaggtggaggtcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 21 - protein, 233 aa, synthetic construct
  REGION 1..233: note = amino acid sequence for the sFLT02 protein
MVSYWDTGVLLCALLSCLLLTGSGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTGGGGGGGGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 1434 bp, Homo sapiens
  misc_feature 1..1434: note = nucleotide sequence of the IgG1 lambda heavy chain
  source 1..1434: mol_type = genomic DNA
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtaattatggcatgcactgggtccgccaggctccaggcaaggggctggagtgggtggcagctatatggtatgatggaagtaataaatactatgcagactccgtgaagggccgattcaccatctccagagacaattccaagaacacgttgtatatgcaaatgaacagcctgagagccgaggacacggctgtgtattattgtgcgagagagggtcggtgggtacgatatactacggtgactactatcggatactactttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 23 - protein, 477 aa, Homo sapiens [sequence omitted]